SEQUENCE LISTING (1 sequence)

SEQ ID NO: 1 - protein, 9 aa, Homo sapiens
LAGLIDADG

(19) United States
(12) Patent Application Publication
(10) Pub. No.: US 2022/0279767 A1
(43) Pub. Date: Sep. 8, 2022

(54) TRANSGENIC SWINE, METHODS OF MAKING AND USES THEREOF, AND METHODS OF MAKING HUMAN IMMUNE SYSTEM MICE

(71) Applicant: The Trustees of Columbia University in the City of New York, New York, NY (US)

(72) Inventors: Megan SYKES, New York, NY (US); Robert J. HAWLEY, New York, NY (US)

(21) Appl. No.: 17/722,697

(22) Filed: Apr. 18, 2022

Related U.S. Application Data
(63) Continuation of application No. PCT/US2020/056771, filed on Oct. 22, 2020.
(60) Provisional application No. 62/925,859, filed on Oct. 25, 2019.
(60) Provisional application No. 62/924,228, filed on Oct. 22, 2019.

(51) Int. Cl. A01K 67/027; A61K 35/26

(52) U.S. Cl. CPC A01K 67/0278; A61K 35/26; A01K 2217/052; A01K 2227/108; A01K 2227/105; A01K 2267/0387; A01K 2267/025; A01K 2207/12

(57) ABSTRACT

The present disclosure provides for transgenic swine, comprising one or more nucleotide sequences encoding one or more HLA I polypeptides and/or one or more HLA II polypeptides inserted into one or more native SLA loci of the swine genome, methods of making and methods of using.

The present disclosure also provides for improved methods of making human immune system mice.

### CROSS-REFERENCE TO OTHER APPLICATIONS

The present application is continuation of International Application No. PCT/US2020/056771, filed on Oct. 22, 2020, which claims priority to U.S. Patent Application Ser. Nos. 62/924,228 filed Oct. 22, 2019 and 62/925,859 filed Oct. 25, 2019, each of which are hereby incorporated by reference in their entireties.

### STATEMENT OF GOVERNMENTAL INTEREST

This invention was made with government support under AI045897 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 16, 2022, is named CU19383—AS FILED—Sequence Listing—01001/007913-US2 and is 1 kilobyte in size.

### FIELD

The present disclosure provides for transgenic swine, comprising one or more nucleotide sequences encoding one or more HLA I polypeptides and/or one or more HLA II polypeptides inserted into one or more native SLA loci of the swine genome, methods of making and methods of using.

The present disclosure also provides for improved methods of making human immune system mice.

### BACKGROUND

Human immune system (HIS) mice have enormous potential for the study of human autoimmune disease, transplantation and infectious disease. A critical tissue needed to produce robust human immune systems in immunodeficient mice is fetal human thymus tissue, which generates a highly functional, diverse repertoire of human T cells. Post-natal human thymus tissue lacks the growth potential to generate large numbers of human T cells that can be generated to become bigger than the murine kidney under whose capsule it is placed. Although some human T cells develop in the native murine thymus in immunodeficient mice, the thymic function is abnormal and disordered and only a small number of human T cells, which do not undergo normal thymic education needed for proper tolerance induction are generated. Therefore human fetal thymic tissue is considered optimal for HIS mouse models. However, the availability of human fetal tissue for research is not a given. Thus, an alternative source of tissue is needed.

Fetal pig thymus tissue can provide that alternative. Fetal swine (SW) thymus (THY) tissue has similar growth characteristics as human (HU) fetal THY tissue when grafted to immunodeficient mice, and supports high levels of robust human thymopoiesis and peripheral immune reconstitution from human CD34+ cells. However, the absence of HLA molecules on SW thymic epithelial cells (TECs) limits the negative selection of conventional T cells and positive selection of regulatory T cells that recognize HLA-restricted antigen (TRAs) produced by the TECs. It also limits the positive selection of human T cells that can recognize foreign antigens in the context of an individual's HLA. Thus, improvement is needed when using the fetal swine thymus tissue to generate HIS mice. Additionally, there is a need for improvement when using swine thymus tissue for other indications such as xenotransplantation to humans.

Described herein is an improved method of producing a human immune system mouse using fetal swine thymus tissue. Also described herein is a transgenic swine.

### SUMMARY

Provided herein are transgenic swine, methods of generating such swine, and uses of such swine.

In one embodiment, the transgenic swine comprises one or more nucleotide sequences encoding one or more HLA I polypeptides and/or one or more HLA II polypeptides inserted into one or more native SLA loci of the swine genome.

In some embodiments, the human HLA is selected from the group consisting of HLAI polypeptides and HLAII polypeptides. In some embodiments, the human HLA1 is selected from the group consisting of HLA-A, HLA-B, HLA-C, HLA-E, HLA-F and HLA-G. In some embodiments, the HLAI polypeptide is HLA-A2.

In some embodiments, the HLA II polypeptides are selected from the group consisting of HLA-DP, HLA-DM, HLA-DO, HLA-DQ, and HLA-DR. In some embodiments, the HLA II polypeptide is HLA-DQ8 or SLA-DRa. In some embodiments, the HLA-DQ8 polypeptides are targeted to the native SLA-DQa locus through a bicistronic vector encoding HLA-DQ8 (HLA-DQA1:03:01:01 and HLA-DQB1:03:02:01).

In some embodiments, the native SLA locus is SLA-1, SLA-2 or SLA-3. In some embodiments, the SLA locus is the SLA-DQα or SLA-DR□ locus. In some embodiments, the nucleic acid is inserted or integrated behind the native SLA promoter. In some embodiments, the nucleic acid encoding the HLA polypeptide is inserted or integrated at the intron 1/exon 2 junction of the native SLA locus.

In some embodiments, the nucleic acid encoding the HLA polypeptide is inserted or integrated into the native SLA locus using a targeting vector. In some embodiments, the vector is bicistronic. In some embodiments, the vector is promoterless.

In some embodiments, the vector further comprises a high efficiency IRES element.

In some embodiments, the vector further comprises polyadenylation site. In some embodiments, the polyadenylation site is a rabbit β-globin.

Also provided for herein are methods of generating, and uses of, the transgenic swine, including but not limited to xenotransplantation into human subjects.

Provided herein is are improved methods for generating human immune system mice.

In some embodiments, the method comprises thymectomizing the mouse and introducing porcine fetal thymic tissue and human CD34+ cells into the mouse. In some embodiments, the human CD34+ cells are derived from cord blood.

In some embodiments, the method comprises thymectomizing the mouse and introducing porcine fetal thymic tissue from a transgenic swine as described herein.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1. Multigenic insertion into the Sachs Miniature Swine GGTA1 locus. FIG. 1A is a schematic of a 10.5 kbp transgene cassette inserted via CRIPSR-assisted homologous recombination between identical genomic targeting arm segments (blue). The cassette contains two bicistronic units, linked by self-splicing 2A elements (yellow), both driven by the ubiquitously expressed CAG promoter. FIG. 1B are the results of FCM analysis of peripheral blood lymphocytes from a cloned transgenic pig (right hand peak) and a non-transgenic control (left hand peak).

FIG. 2. Targeted insertion of a bicistronic cassette encoding the human IL3 receptor behind the native pig ILRa promoter. FIG. 2A shows the genomic region downstream of the IL3Ra gene (top). Exons 2 through the pA site of the IL3Ra gene are shown in blue. Exons 2 through the pA site of the SLC25A6 gene are shown in red. The targeting vector for addition of the human IL3Ra and IL3Rb chains is shown at the bottom. Homologous recombination between the genomic identical sequences (solid blue and red) results in the replacement of 15.7 kbp of native genomic sequence, including most of the native IL3Ra gene, with 7.1 kbp of sequence encoding the human IL3R chains and tagging the end of the SLC25A6 gene (via a T2A element) with a GFP CDS (green). FIG. 2B shows the second round of flow sorting of fetal fibroblasts transfected with the promoter trap vector. Low GFP fluorescent cells (white) and high fluorescent cells (yellow) were recovered separately. FIG. 2C are the results of targeting analysis of flow sorted populations. PCR was performed at the upstream and downstream ends of genomic DNA using primer pairs that included one primer outside the vector sequence generated bands indicating proper targeting of the upstream end in both the low and high fluorescent fractions, while PCR at the downstream end generated the expected size band only in the high fluorescent population. FIG. 2D shows the results of targeting analysis of genomic DNA of 8 day 39 fetuses generated by SCNT with cells from the high fluorescence sorted population. All 8 fetuses generated bands indicative of proper targeting at both the upstream (US) and downstream (DS) ends. FIG. 2E are the results of RT-PCR analysis of gene expression in liver cells from the 8 transgenic fetuses. As expected, all 8 fetuses produced a transcript from the recombinant SLC25A6-GFP gene. All 8 also produced a properly spliced transcript from the human IL3Ra-IRES-IL3Rb cassette.

FIG. 3 shows the HLA-A2 targeting of an SLA I gene. The top schematic is the native gene. The bottom schematic is the promoterless targeting vector. Recombination, enhanced by paired CRISPR/Cas9 nicks near the SLA intron1/exon 2 junction of the native locus, with the promoterless targeting vector results in the addition of a cassette comprised of the mature form of human B2 microglobulin fused to the mature coding sequences of HLA-A2 (A*02:01). The leader peptide for the fusion protein is provided by SLA1 Exon 1 and the resulting transcript terminated at a rabbit β-globin polyadenylation site. Due to the promoterless design of the vector, a very high proportion of cells expressing the human human B2m/HLA-A2 fusion will be properly target the DQA gene.

FIG. 4 shows the results of flow cytometry of cells stained with pan-haplotype anti-pig DR or anti-pig DQ antibody after 6 days of culture with IFN-g (right curve) or without IFN-g (left curve).

FIG. 5 shows the HLA-DQ8 targeting of the SLA-DQA gene. The top schematic is the native gene. The bottom schematic is the promoterless targeting vector. Recombination, enhanced by paired CRISPR/Cas9 nicks near the DRA intron1/exon 2 junction of the native locus, with the promoterless targeting vector results in the addition of a cassette comprised of the mature form of human DQ8α (DQA* 03:01), an IRES element and the precursor form of DQ8β (DQB1*03:02), terminating with a rabbit β-globin polyadenylation site. Due to the promoterless design of the vector, a very high proportion of cells expressing the human DQ8α and DQ8β will properly target the DQA gene.

FIG. 6 shows the study showing the importance of HLA sharing between the thymus and peripheral APCs for human T cell homeostasis in HIS mice. FIG. 6A is a schematic of the experimental design. FIG. 6B is a graph of the proportion of proliferating (Ki67+) T cells in each type of mice 10 day post adoptive transfer.

FIG. 7 show the comparison of human immune reconstitution in various HIS mice. FIG. 7A is a graph of the numbers of human CD3+ cells in the peripheral blood of the indicated mice at the indicated times post transfer. FIG. 7B is flow cytometry analysis showing the phenotype of T cells from a representative mouse at week 15 post-transplantation.

FIG. 8 show the positive selection for MART1 TCR in HLA-A2+ human thymus but not in swine thymus. CD34 cells were lentivirally transduced with GFP-MART1 TCR and injected into thymectomized NSG mice receiving the indicated THY grafts. The graph shows the reduced numbers of GFP+MART1+ TCR+(detected with MART1 tetramer) thymoctyes in SW and HLA-A2-negative HU THY grafts compared to HLA-A2+HU THY grafts.

FIG. 9 shows evidence of HLA-restricted TCR, Clone 5 (specific for insulin B 9-23 presented by HLA-DQ8), when introduced into human hematopoietic stem cells, is positively selected in an HLA-DQ8 human thymus in HIS mice but negatively selected only if the hematopoietic stem cells express HLA-DQ8. HLA-DQ8 Tg NSG mice received HLA-DQ8+ human fetal thymus and HLA-DQ8 or DQ8− fetal liver CD34+ HSCs transduced with Clone 5 TCR. FIG. 9A. shows the absolute numbers of GFP+ Clone 5 CD4/8DP and SP thymocytes were decreased in the thymi of mice receiving DQ8+ compared to DQ8-negative HSCs. FIG. 9B shows enrichment of T cell lineage committed (CD1a+) Clone 5 (GFP+) cells among double negative thymocytes in the thymi of mice receiving DQ8+ compared to DQ8− HSCs.

### DETAILED DESCRIPTION OF THE INVENTION

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The term “isolated” as used herein refers to molecules or biologicals or cellular materials being substantially free from other materials.

As used herein, the term “functional” may be used to modify any molecule, biological, or cellular material to intend that it accomplishes a particular, specified effect.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, “target”, “targets” or “targeting” refers to partial or no breakage of the covalent backbone of polynucleotide. In one embodiment, a deactivated Cas protein (or dCas) targets a nucleotide sequence after forming a DNA-bound complex with a guide RNA. Because the nuclease activity of the dCas is entirely or partially deactivated, the dCas binds to the sequence without cleaving or fully cleaving the sequence. In some embodiment, targeting a gene sequence or its promoter with a dCas can inhibit or prevent transcription and/or expression of a polynucleotide or gene.

The term “Cas9” refers to a CRISPR associated endonuclease referred to by this name Non-limiting exemplary Cas9s are provided herein, e.g., the Cas9 provided for in UniProtKB G3ECR1 (CAS9_STRTR) or the Staphylococcus aureus Cas9, as well as the nuclease dead Cas9, orthologs and biological equivalents each thereof. Orthologs include but are not limited to Streptococcus pyogenes Cas9 (“spCas9”), Cas 9 from Streptococcus thermophiles, Legionella pneumophilia, Neisseria lactamica, Neisseria meningitides, Francisella novicida; and Cpf1 (which performs cutting functions analogous to Cas9) from various bacterial species including Acidaminococcus spp. and Francisella novicida U112.

As used herein, the term “CRISPR” refers to a technique of sequence specific genetic manipulation relying on the clustered regularly interspaced short palindromic repeats pathway. CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. Gene regulation refers to increasing or decreasing the production of specific gene products such as protein or RNA.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, et al. 2014. Nature biotechnology 32(12):1262-7, Mohr, et al. 2016. FEBS Journal 3232-38, and Graham, et al. 2015. Genome Biol. 16:260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, et al. 2016. J of Biotechnology 233:74-83). As used herein, a biological equivalent of a gRNA includes but is not limited to polynucleotides or targeting molecules that can guide a Cas9 or equivalent thereof to a specific nucleotide sequence such as a specific region of a cell's genome.

The term “embryo” refers to the early stage of development of a multicellular organism. In general, in organisms that reproduce sexually, embryonic development refers to the portion of the life cycle that begins just after fertilization and continues through the formation of body structures, such as tissues and organs. Each embryo starts development as a zygote, a single cell resulting from the fusion of gametes (i.e., fertilization of a female egg cell by a male sperm cell). In the first stages of embryonic development, a single-celled zygote undergoes many rapid cell divisions, called cleavage, to form a blastula.

“Transgenic” and its grammatical equivalents as used herein, include donor animal genomes that have been modified to introduce non-native genes from a different species into the donor animal's genome at a non-orthologous, non-endogenous location such that the homologous, endogenous version of the gene (if any) is retained in whole or in part. “Transgene,” “transgenic,” and grammatical equivalents as used herein do not include reprogrammed genomes, knock-outs or other modifications as described herein.

“Tolerance”, as used herein, refers to the inhibition or decrease of a graft recipient's ability to mount an immune response, e.g., to a donor antigen, which would otherwise occur, e.g., in response to the introduction of a non self MHC antigen into the recipient. Tolerance can involve humoral, cellular, or both humoral and cellular responses. The concept of tolerance includes both complete and partial tolerance. In other words, as used herein, tolerance include any degree of inhibition of a graft recipient's ability to mount an immune response, e.g., to a donor antigen.

“Hematopoietic stem cell”, as used herein, refers to a cell that is capable of developing into mature myeloid and/or lymphoid cells. Preferably, a hematopoietic stem cell is capable of the long-term repopulation of the myeloid and/or lymphoid lineages. Stem cells derived from the cord blood of the recipient or the donor can be used in methods of the disclosure.

“Miniature swine”, as used herein, refers to completely or partially inbred miniature swine.

“Graft”, as used herein, refers to a body part, organ, tissue, cells, or portions thereof.

### Abbreviations

SW—swine
HU—human
TEC—thymic epithelial cells
TMC—thymic mesenchyme cells
WBC—white blood cells
DP—double positive cells (both CD4+, CD8+)
SP—single positive cells (either CD4+ or CD8+)
Tregs—regulatory T cells
LN—lymph nodes
TRA—tissue restricted antigens
HSCs—human hematopoietic cells
NSG—NOD scid common γ chain knockout
SCNT—somatic cell nuclear transfer

The current disclosure provides for transgenic swine pig comprising a nucleotide sequence encoding an HLA I or HLA II polypeptide inserted into the SLA locus of the pig genome, methods of generating such transgenic swine, and methods of using such transgenic swine.

The current disclosure also provides for human immune system (HIS) mice generated using thymus from the transgenic fetal swine as well as human immunized mice generated using thymus from fetal swine and CD34+ cells from cord blood, and methods of generating such HIS mice.

### Transgenic Swine

The inventors have previously shown that robust human thymopoiesis occurs in porcine thymus grafts (Nikolic, et al. 1999; Shimizu, et al. 2008; Kalscheuer, et al. 2014). However, peripheral human T cells that were generated in a pig compared to a human fetal thymus show subtle impairments in HLA-restricted immune functions and homeostasis and tolerance to tissue restricted antigens. The addition of transgenic HLA molecules to the porcine thymus tissue could overcome most of these limitations. Thus, disclosed herein are several strains of transgenic pigs that express common HLA alleles in place of some swine leukocyte antigen (SLA, the pig counterpart of HLA) molecules. These transgenic swine can be used as a source of thymus tissue for many purposes, including generating HIS mice and as donor tissue. Transgenic expression of common HLA molecules will improve positive selection of HLA-restricted human T cells and generation of functional regulatory T (Treg) cells that interact effectively with human antigen-presenting cells (APCs) in the periphery and will improve negative selection of human TRA-reactive T cells, thereby reducing the risk of autoimmunity.

Baboons receiving porcine thymokidney grafts have shown evidence of de novo recipient (baboon) thymopoiesis in the porcine thymic graft, appearance of recent thymic emigrants in the periphery and donor-specific unresponsiveness in Elispot and MLR assays, as well as a decline in non-Gal natural antibodies. While the latter may reflect absorption by the pig kidney, minimal IgM binding was detected on these xenografts, with no complement fixation or significant pathology. Thus, the results obtained with this model demonstrate the potential of composite thymus-kidney xenografts to induce tolerance in primates.

Limitations of generating a human T cell repertoire in a xenogeneic porcine thymus include the preferential recognition of microbial antigens on porcine MHC, which would be useful for protecting the graft but would not optimize protection against microbial pathogens infecting the host, as well as the failure to negatively select conventional T cells and positively select Tregs recognizing human tissue-restricted antigens (TRAs). Indeed, studies in humanized mice have shown reduced responses to peptides presented by human APCs following immunization when the human T cells developed in a pig rather than a human thymus graft.

One approach to overcome this limitation involves creation of a “hybrid thymus”, in which recipient thymic epithelial cells obtained either from thymectomy specimens or generated from stem cells are injected into the porcine thymic tissue. Hybrid thymi from post-natal thymus donors have been generated, where the hybrid thymus promotes tolerance to human TRAs among human T cells.

Pig thymus grafts have been shown to support the development of normal, diverse murine or human T cell repertoires and these T cells are specifically tolerant of the xenogeneic pig donor. However, recognition of foreign antigens presented by recipient HLA molecules in the periphery is suboptimal. Thus, immune function may be less than optimal. As previously shown in co-owned application no. PCT/US2019/0051865, this can be overcome by providing recipient TECs in the pig-human hybrid thymus graft because these TECs will participate in positive selection, resulting in T cells that can more readily recognize foreign antigens presented by recipient HLA molecules in the periphery. For pig thymus grafts, survival, homeostasis and function of T cells that do not find their “positive selecting” ligand in the periphery is suboptimal. The positive selecting ligand is the MHC/peptide complex on TECs that rescue thymocytes from programmed cell death when the thymocyte has a low affinity T cell receptor recognizing that complex. Providing recipient TECs in the pig-human hybrid thymus allows positive selection of T cells that will find the same ligand on recipient cells in the periphery, conferring normal survival, homeostasis and function. This use of a hybrid thymus instead of a simple pig thymus can improve the function and self-tolerance of a human T cell repertoire generated in a pig thymus while allowing tolerance to the pig to develop. It follows that the use of transgenic swine thymus can also improve the function and self-tolerance of a human T cell repertoire generated in a pig thymus. Thus, the transgenic swine disclosed here can also be used a source for donor thymus tissue.

The Sachs miniature swine colony was established from two founder animals by Dr. David Sachs in the 1970s. The MHC (Swine Leukocyte Antigens, SLA) of these animals were defined serologically by Dr. Sachs and 3 SLA-homozygous partially inbred lines have been maintained, along with a number of intra-SLA recombinants. These swine can be the source animals of the transgenic pig disclosed herein (U.S. Pat. No. 6,469,229 (Sachs), U.S. Pat. No. 7,141,716 (Sachs), each of the disclosures of which are incorporated by reference herein). The creation of such swine through the described methods, and/or the utilization of such swine and progeny following creation, can be employed in the practice of the present disclosure, including, but not limited to, utilizing organs, tissue and/or cells derived from such swine.

In some embodiments, cells from the swine are the starting material. In some embodiments, the cells are fibroblasts. In some embodiments, the cells are from GTA1 null, SLA haplotype h homozygous Sachs Miniature Swine (SLA-1*02:01, SLA-1*07:01, SLA-2*02:01, SLA-3 null, SLA-DRA*01:01:02, SLA-DRB*02:01, SLA-DQA*02:02:01, SLADQB*04:01:01). Due to the partially inbred nature of these animals, offspring will have a high degree of genetic similarity.

In some embodiments, cells which have been previously modified by the insertion or integration of a nucleic acid sequence encoding the HLA polypeptides into the native SLA locus is the starting material.

In the human, major histocompatibility complex (MHC) molecules are referred to as HLA, an acronym for human leukocyte antigens, and are encoded by the chromosome 6p21.3-located HLA region. The HLA segment is divided into three regions (from centromere to telomere), Class II, Class III and Class I. These cell-surface proteins are responsible for the regulation of the immune system in humans. HLA genes are highly polymorphic, which means that they have many different alleles, allowing them to fine-tune the adaptive immune system. The proteins encoded by certain genes are also known as antigens, as a result of their historic discovery as factors in organ transplants. Different classes have different functions.

HLAs corresponding to MHC class I (A, B, and C) which all are the HLA Class1 group present peptides from inside the cell. In general, these particular peptides are small polymers, about 9 amino acids in length. Foreign antigens presented by MHC class I attract killer T-cells (also called CD8 positive- or cytotoxic T-cells) that destroy cells. MHC class I proteins associate with β2-microglobulin, which unlike the HLA proteins is encoded by a gene on chromosome 15.

HLAs corresponding to MHC class II (DP, DM, DO, DQ, and DR) present antigens from outside of the cell to T-lymphocytes. These particular antigens stimulate the multiplication of T-helper cells (also called CD4 positive T cells), which in turn stimulate antibody-producing B-cells to produce antibodies to that specific antigen. Self-antigens are suppressed by regulatory T cells. The affected genes are known to encode 4 distinct regulatory factors controlling transcription of MHC class II genes.

HLAs corresponding to MHC class III encode components of the complement system.

Aside from the genes encoding the 6 major antigen-presenting proteins, there are a large number of other genes, many involved in immune function, located on the HLA complex.

Diversity of HLAs in the human population is one aspect of disease defense, and, as a result, the chance of two unrelated individuals with identical HLA molecules on all loci is extremely low. HLA genes have historically been identified as a result of the ability to successfully transplant organs between HLA-similar individuals.

Each human cell expresses six MHC class I alleles (one HLA-A, -B, and -C allele from each parent) and six to eight MHC class II alleles (one HLA-DP and -DQ, and one or two HLA-DR from each parent, and combinations of these). The MHC variation in the human population is high, at least 350 alleles for HLA-A genes, 620 alleles for HLA-B, 400 alleles for DR, and 90 alleles for DQ. In humans, MHC class II molecules are encoded by three different loci, HLA-DR, -DQ, and -DP, which display about.70% similarity to each other. Polymorphism is a notable feature of MHC class II genes. This genetic diversity presents problems during xenotransplantation where the recipient's immune response is the most important factor dictating the outcome of engraftment and survival after transplantation.

In some embodiments, the present disclosure includes modifying a swine by the insertion or integration of a nucleic acid encoding one or more human HLA polypeptides into one or more native SLA loci of the swine.

In some embodiments, the human HLA is selected from the group consisting of HLA1 polypeptides and HLAII polypeptides. In some embodiments, the human HLA1 is selected from the group consisting of HLA-A, HLA-A2, HLA-B, HLA-C, HLA-E, HLA-F and HLA-G. In some embodiments, the HLAI polypeptide is HLA-A2. In some embodiments, the HLA II polypeptides are selected from the group consisting of HLA-DP, HLA-DM, HLA-DO, HLA-DQ, and HLA-DR. In some embodiments, the HLA II polypeptide is HLA-DQ8.

In some embodiments, the human HLA is a known HLA polypeptide. Such HLA sequences are available, e.g., in the IPD-IMGT/HLA database (available at ebi.ac.uk/ipd/imgt/hla/) and the international ImMunoGeneTics information System® (available at imgt.org). For example, HLA-A1, B8, DR17 is the most common HLA haplotype among Caucasians, with a frequency of 5%. Thus, the disclosed method can be performed using the known HLA sequence information in combination with the methods described herein.

In some embodiments, the nucleic acid encoding the human HLA polypeptide is derived from a specific human individual. In some embodiments, the transgenic swine is produced using the nucleic acid encoding the human HLA polypeptide derived from the specific human individual and thymic tissue or other cells, tissues or organs from the transgenic swine will be introduced into the same specific human individual. In these embodiments, a human leukocyte antigen (HLA) gene from the specific human individual who will receiving a xenotransplantion from the transgenic swine are identified and sequenced. It will be understood that identifying and sequencing a particular HLA allele can be done by methods known in the art.

The known human HLA sequence or identified and sequenced HLA sequence(s) from a specific human individual may be introduced into a vector under the control of a SLA promoter e.g., to have 90%, 95%, 98%, 99%, or 100% sequence homology to the HLA sequence.

In some embodiments, the nucleic acid encoding the HLA polypeptide can be optimized to have the sequence of the HLA polypeptide or mimic the HLA alleles of a recipient mammal.

In some embodiments, the HLA polypeptide is fused to another protein. In some embodiments, the protein is human β-2 microglobulin (B2M). In some embodiments, an HLA-A2 is fused to a B2M. Introduction of HLA-A2 and human B2m as a fusion protein will ensure that heterotypic interactions between HLA-A2 and pig B2m will not interfere with HLA-A2 surface expression.

In some embodiments, the native SLA locus is SLAI. In some embodiments, the native SLA locus is SLA-1 or SLA-2. In some embodiments, the SLA locus is the SLA-DQα locus. In some embodiments, the nucleic acid is inserted or integrated behind the native SLA promoter. In some embodiments, the nucleic acid encoding the HLA polypeptide is inserted or integrated at the intron 1/exon 2 of the native SLA locus.

In some embodiments, the nucleic acid encoding the HLA polypeptide is inserted or integrated into the native SLA locus using a targeting vector. In some embodiments, the vector is bicistronic. In some embodiments, the vector is promoterless. The use of a promoterless design of the vector ensures that a very high proportion of cells expressing the human B2m/HLA-A2 fusion will be properly target the DQA gene.

In some embodiments, the vector further comprises a high efficiency IRES element.

In some embodiments, the vector further comprises polyadenylation site. In some embodiments, the polyadenylation site is a rabbit β-globin.

Methods of modifying the SLA locus by the integration or insertion of nucleic acids encoding HLA polypeptides include the use of site specific nucleases as described below.

Thus provided herein are methods of generating transgenic swine. In one aspect, a specific human individual recipient's HLA gene is sequenced and used in the targeting vector construction for introduction into the swine cells. In another aspect, a known human HLA genotype from a WHO database may be used in the targeting vector construction for introduction into the swine cells. A targeting vector as described herein is constructed using the nucleic acid encoding the HLA polypeptide. CRISPR-Cas9 plasmids can be prepared. CRISPR cleavage sites at the SLA/MHC locus in the swine cells are identified and gRNA sequences targeting the cleavage sites designed and are cloned into one or more CRISPR-Cas9 plasmids. CRISPR-Cas9 plasmids are then administered into the swine cells along with the targeting vectors.

Once the modification has been completed, the cells are screened for the desired modification using methods known in the art. The cells with the desired modification can be used as somatic cell nuclear transfer (SCNT) donor cells for nuclear transfer/embryo transfer and production of transgenic swine fetuses and piglets, also by methods know in the art.

Transgenic swine fetuses are harvested at approximately 40 weeks. These fetuses will be analyzed for expression and proper integration of the desired HLA gene. Fetuses that are found to have the proper integration are used as the source of cell lines for SCNT cloning for generating additional fetuses and piglets. Fetuses are harvested at approximately 56-70 weeks for thymic isolation.

The fetuses will also be used to generate transgenic founder boars.

Thymic tissue from the transgenic fetal swine has many uses including but not limited to the generation of an improved human immune system (HIS) mouse as described below.

The cells, tissue and/or organs from the transgenic fetal swine, including thymic tissue, can also be used for xenotransplantation as well as recovering or restoring impairment of the function of the thymus and reconstituting T cells in a subject. In some embodiments, the subject is a mammal. In some embodiments, the subject is a human

Cells, tissues, and organs for purposes of xenotransplantation derived from the transgenic swine will have reduced rejection as compared to cells, tissues, and organs derived from a wild-type swine.

Also encompassed by the present disclosure is a method of xenotransplantation in a recipient mammal of a first species, the method comprising introducing thymic tissue into the recipient mammal, wherein the thymic tissue is from a transgenic swine described herein.

The present disclosure also provides for a method of restoring or inducing immunocompetence in a recipient mammal of a first species, the method comprising the step of introducing a thymic tissue into the recipient mammal, wherein the thymic tissue is from a transgenic swine described herein.

The present disclosure also provides for a method of restoring or promoting thymus-dependent ability for T cell progenitors to develop into mature functional T cells in a recipient mammal of a first species, the method comprising introducing thymic tissue into the recipient mammal of the first species, wherein the thymic tissue is from a transgenic swine described herein.

In one embodiment, thymic function is essentially absent in the recipient mammal before thymic tissue is introduced. In another embodiment, the recipient mammal is thymectomized before thymic tissue is introduced. In yet another embodiment, the recipient mammal has an immune disorder.

The second species may be swine, such as a transgenic swine.

The first species may be primate, such as non-human primate or human.

In one embodiment, the recipient mammal is a human and the donor mammal is a transgenic swine described herein. In some embodiments, the recipient human is the source of the nucleic acid encoding the HLA polypeptides that is introduced into the swine to generate the transgenic swine. In some embodiments, the nucleic acid encoding the HLA polypeptide is one known in the art.

In one embodiment, the thymic tissue is implanted in the recipient mammal. For example, the thymic tissue may be implanted as a primarily vascularized thymus lobe or composite thymo-kidney graft. The thymic tissue may be transplanted intramuscularly in the recipient. The thymic tissue may be transplanted either into the quadriceps muscle alone or with additional transplantation sites (e.g., kidney capsule and omentum) in the recipient.

### CRISPR/Cas and Other Endonucleases

Any suitable nuclease may be used in the present methods to produce the transgenic swine. Nucleases are enzymes that hydrolyze nucleic acids. Nucleases may be classified as endonucleases or exonucleases. An endonuclease is any of a group of enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyses the hydrolysis of RNA may be referred to as a ribonuclease or an RNase. Some nucleases are specific to either single-stranded or double-stranded nucleic acid sequences. Some enzymes have both exonuclease and endonuclease properties. In addition, some enzymes are able to digest both DNA and RNA sequences.

Non-limiting examples of the endonucleases include a zinc finger nuclease (ZFN), a ZFN dimer, a ZFNickase, a transcription activator-like effector nuclease (TALEN), or a RNA-guided DNA endonuclease (e.g., CRISPR/Cas). Meganucleases are endonucleases characterized by their capacity to recognize and cut large DNA sequences (12 base pairs or greater). Any suitable meganuclease may be used in the present methods to create double-strand breaks in the host genome, including endonucleases in the LAGLIDADG and PI-Sce family

One aspect of the present disclosure provides RNA-guided endonucleases. RNA-guided endonucleases also comprise at least one nuclease domain and at least one domain that interacts with a guide RNA. An RNA-guided endonuclease is directed to a specific nucleic acid sequence (or target site) by a guide RNA. The guide RNA interacts with the RNA-guided endonuclease as well as the target site such that, once directed to the target site, the RNA-guided endonuclease is able to introduce a double-stranded break into the target site nucleic acid sequence. Since the guide RNA provides the specificity for the targeted cleavage, the endonuclease of the RNA-guided endonuclease is universal and can be used with different guide RNAs to cleave different target nucleic acid sequences.

One example of a RNA guided sequence-specific nuclease system that can be used with the methods and compositions described herein includes the CRISPR system (Wiedenheft, et al. 2012 Nature 482:331-338; Jinek, et al. 2012 Science 337:816-821; Mali, et al. 2013 Science 339:823-826; Cong, et al. 2013. Science 339:819-823). The CRISPR (Clustered Regularly Interspaced Short Palindromic Repeats) system exploits RNA-guided DNA-binding and sequence-specific cleavage of target DNA. The guide RNA/Cas combination confers site specificity to the nuclease. A single guide RNA (sgRNA) contains about 20 nucleotides that are complementary to a target genomic DNA sequence upstream of a genomic PAM (protospacer adjacent motifs) site (e.g., NGG) and a constant RNA scaffold region. The Cas (CRISPR-associated) protein binds to the sgRNA and the target DNA to which the sgRNA binds and introduces a double-strand break in a defined location upstream of the PAM site. Cas9 harbors two independent nuclease domains homologous to HNH and RuvC endonucleases, and by mutating either of the two domains, the Cas9 protein can be converted to a nickase that introduces single-strand breaks (Cong, et al. 2013 Science 339:819-823). It is specifically contemplated that the methods and compositions of the present disclosure can be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism. The nuclease can be introduced into the cell in form of a DNA, mRNA and protein.

It is appreciated by those skilled in the art that gRNAs can be generated for target specificity to target a specific gene, optionally a gene associated with a disease, disorder, or condition. Thus, in combination with Cas9, the guide RNAs facilitate the target specificity of the CRISPR/Cas9 system. Further aspects such as promoter choice, may provide additional mechanisms of achieving target specificity, e.g., selecting a promoter for the guide RNA encoding polynucleotide that facilitates expression in a particular organ or tissue. Accordingly, the selection of suitable gRNAs for the particular disease, disorder, or condition is contemplated herein. In one embodiment, the gRNA hybridizes to a gene or allele that comprises a single nucleotide polymorphism (SNP).

Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In one embodiment, the RNA-guided endonuclease is derived from a type II CRISPR/Cas system. In specific embodiments, the RNA-guided endonuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum the rmopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In some embodiments, the nucleotide sequence encoding the Cas (e.g., Cas9) nuclease is modified to alter the activity of the protein. In some embodiments, the Cas (e.g., Cas9) nuclease is a catalytically inactive Cas (e.g., Cas9) (or a catalytically deactivated/defective Cas9 or dCas9). In one embodiment, dCas (e.g., dCas9) is a Cas protein (e.g., Cas9) that lacks endonuclease activity due to point mutations at one or both endonuclease catalytic sites (RuvC and HNH) of wild type Cas (e.g., Cas9). For example, dCas9 contains mutations of catalytically active residues (D10 and H840) and does not have nuclease activity. In some cases, the dCas has a reduced ability to cleave both the complementary and the non-complementary strands of the target DNA. In some cases, the dCas9 harbors both D10A and H840A mutations of the amino acid sequence of S. pyogenes Cas9. In some embodiments when a dCas9 has reduced or defective catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the Cas protein can still bind to target DNA in a site-specific manner, because it is still guided to a target polynucleotide sequence by a DNA-targeting sequence of the subject polynucleotide (e.g., gRNA), as long as it retains the ability to interact with the Cas-binding sequence of the subject polynucleotide (e.g., gRNA).

Inactivation of Cas endonuclease activity can create a catalytically deactivated Cas (dCas, e.g., dCas9). dCas can bind but not cleave DNA, thus preventing the transcription of the target gene by creating a physical barrier to the action of transcription factors. This rendition of CRISPR works at the transcription level in a reversible fashion. This strategy has been termed CRISPR interference, or CRISPRi. In CRISPR interference (CRISPRi), dCas fusion proteins (e.g., dCas fused to another protein or portion thereof) may be used in the presently disclosed methods. In some embodiments, dCas is fused to a (transcriptional) repressor domain or a transcriptional silencer. Non-limiting examples of transcriptional repression domains include a Krüppel-associated Box (KRAB) domain, an ERF repressor domain (ERD), a mSin3A interaction domain (SID) domain, concatemers of SID (e.g. SID4X), or a homolog thereof. Non-limiting examples of transcriptional silencers include Heterochromatin Protein 1 (HP1). CRISPRi may be modified by fusing Cas (e.g., dCas) to the Kruppel-associated box repression domain (KRAB), which augments the repressive effects of Cas. Gilbert et al. 2013. Cell 154(2):442-51.

Second generation CRISPRi strongly represses via PUF-KRAB repressors. PUF proteins (named after Drosophila Pumilio and C. elegans fern-3 binding factor) are known to be involved in mediating mRNA stability and translation. These proteins contain a unique RNA-binding domain known as the PUF domain. The RNA-binding PUF domain, such as that of the human Pumilio 1 protein (referred here also as PUM), contains 8 repeats (each repeat called a PUF motif or a PUF repeat) that bind consecutive bases in an anti-parallel fashion, with each repeat recognizing a single base, i.e., PUF repeats R1 to R8 recognize nucleotides N8 to N1, respectively. For example, PUM is composed of eight tandem repeats, each repeat consisting of 34 amino acids that folds into tightly packed domains composed of alpha helices. PUF and its derivatives or functional variants are programmable RNA-binding domains that can be used in the present methods and systems, as part of a PUF domain-fusion that brings any effector domain to a specific PUF-binding sequence on the subject polynucleotide (e.g., gRNA).

The present methods may use CRISPR deletion (CRISPRd). CRISPRd capitalizes on the tendency of DNA repair strategies to default towards NHEJ and does not require a donor template to repair the cleaved strand. Instead, Cas creates a DSB in the gene harboring a mutation first, then NHEJ occurs, and insertions and/or deletions (INDELs) are introduced that corrupt the sequence, thus either preventing the gene from being expressed or proper protein folding from occurring. This strategy may be particularly applicable for dominant conditions, in which case knocking out the mutated, dominant allele and leaving the wild type allele intact may be sufficient to restore the phenotype to wild type.

In certain embodiments, the Cas enzyme may be a catalytically defective Cas (e.g., Cas9) or dCas, or a Cas nickase or nickase.

The Cas enzyme (e.g., Cas9) may be modified to function as a nickase, named as such because it “nicks” the DNA by inducing single-strand breaks instead of DSBs. The term “Cas nickase” or “nickase”, as used herein, refers to a Cas protein that is capable of cleaving only one strand of a duplexed nucleic acid molecule (e.g., a duplexed DNA molecule). In some embodiments, a Cas nickase may be any of the nickase disclosed in U.S. Pat. No. 10,167,457, the content of which is incorporated herein by reference in its entirety. In one embodiment, a Cas (e.g., Cas9) nickase has an active HNH nuclease domain and is able to cleave the non-targeted strand of DNA, i.e., the strand bound by the gRNA. In one embodiment, a Cas (e.g., Cas9) nickase has an inactive RuvC nuclease domain and is not able to cleave the targeted strand of the DNA, i.e., the strand where base editing is desired. In some embodiments the Cas nickase cleaves the target strand of a duplexed nucleic acid molecule, meaning that the Cas nickase cleaves the strand that is base paired to (complementary to) a gRNA (e.g., an sgRNA) that is bound to the Cas. In some embodiments, the Cas nickase cleaves the non-target, non-base-edited strand of a duplexed nucleic acid molecule, meaning that the Cas nickase cleaves the strand that is not base paired to a gRNA (e.g., an sgRNA) that is bound to the Cas. Additional suitable Cas9 nickases will be apparent to those of skill in the art based on this disclosure and knowledge in the field, and are within the scope of this disclosure.

In CRISPR activation (CRISPRa), dCas may be fused to an activator domain, such as VP64 or VPR. Such dCas fusion proteins may be used with the constructs described herein for gene activation. In some embodiments, dCas is fused to an epigenetic modulating domain, such as a histone demethylase domain or a histone acetyltransferase domain. In some embodiments, dCas is fused to a LSD1 or p300, or a portion thereof. In some embodiments, the dCas fusion is used for CRISPR-based epigenetic modulation. In some embodiments, dCas or Cas is fused to a Fok1 nuclease domain. In some embodiments, Cas or dCas fused to a Fok1 nuclease domain is used for genome editing. In some embodiments, Cas or dCas is fused to a fluorescent protein (e.g., GFP, RFP, mCherry, etc.). In some embodiments, Cas/dCas proteins fused to fluorescent proteins are used for labeling and/or visualization of genomic loci or identifying cells expressing the Cas endonuclease. In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains.

In addition to well characterized CRISPR-Cas system, a new CRISPR enzyme, called Cpf1 (Cas protein 1 of PreFran subtype) may be used in the present methods and systems (Zetsche et al. 2015. Cell). Cpf1 is a single RNA-guided endonuclease that lacks tracrRNA, and utilizes a T-rich protospacer-adjacent motif. The authors demonstrated that Cpf1 mediates strong DNA interference with characteristics distinct from those of Cas9. Thus, in one embodiment of the present invention, CRISPR-Cpf1 system can be used to cleave a desired region within the targeted gene.

In further embodiment, the nuclease is a transcription activator-like effector nuclease (TALEN). TALENs contains a TAL effector domain that binds to a specific nucleotide sequence and an endonuclease domain that catalyzes a double strand break at the target site (PCT Patent Publication No. WO2011072246; Miller et al., 2011 Nat. Biotechnol. 29:143-148; Cermak et al., 2011 Nucleic Acid Res. 39:e82). Sequence-specific endonucleases may be modular in nature, and DNA binding specificity is obtained by arranging one or more modules. Bibikova et al., 2001 Mol. Cell. Biol. 21:289-297; Boch et al., 2009 Science 326:1509-1512.

ZFNs can contain two or more (e.g., 2-8, 3-6, 6-8, or more) sequence-specific DNA binding domains (e.g., zinc finger domains) fused to an effector endonuclease domain (e.g., the Fok1 endonuclease). Porteus et al., 2005 Nat. Biotechnol, 23:967-973; Kim et al., 2007 Proceedings of the National Academy of Sciences of USA, 93:1156-1160; U.S. Pat. No. 6,824,978; PCT Publication Nos. WO1995/09233 and WO1994018313.

In one embodiment, the nuclease is a site-specific nuclease of the group or selected from the group consisting of omega, zinc finger, TALEN, and CRISPR/Cas.

The sequence-specific endonuclease of the methods and compositions described here can be engineered, chimeric, or isolated from an organism. Endonucleases can be engineered to recognize a specific DNA sequence, by, e.g., mutagenesis. Seligman et al. 2002 Nucleic Acids Research 30:3870-3879. Combinatorial assembly is a method where protein subunits form different enzymes can be associated or fused. Arnould et al. 2006 Journal of Molecular Biology 355:443-458. In certain embodiments, these two approaches, mutagenesis and combinatorial assembly, can be combined to produce an engineered endonuclease with desired DNA recognition sequence.

The sequence-specific nuclease can be introduced into the cell in the form of a protein or in the form of a nucleic acid encoding the sequence-specific nuclease, such as an mRNA or a cDNA. Nucleic acids can be delivered as part of a larger construct, such as a plasmid or viral vector, or directly, e.g., by electroporation, lipid vesicles, viral transporters, microinjection, and biolistics. Similarly, the construct containing the one or more transgenes can be delivered by any method appropriate for introducing nucleic acids into a cell.

Guide RNA(s) used in the methods of the present disclosure can be designed so that they direct binding of the Cas-gRNA complexes to pre-determined cleavage sites in a genome. In one embodiment, the cleavage sites may be chosen so as to release a fragment or sequence that contains a region of a frame shift mutation. In further embodiment, the cleavage sites may be chosen so as to release a fragment or sequence that contains an extra chromosome.

For Cas family enzyme (such as Cas9) to successfully bind to DNA, the target sequence in the genomic DNA can be complementary to the gRNA sequence and may be immediately followed by the correct protospacer adjacent motif or “PAM” sequence. “Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule, which can form hydrogen bonds (e.g., Watson-Crick base pairing) with a second nucleic acid sequence. Full complementarity is not necessarily required, provided there is sufficient complementarity to cause hybridization and promote formation of a CRISPR complex. A target sequence may comprise any polynucleotide, such as DNA or RNA polynucleotides. The Cas9 protein can tolerate mismatches distal from the PAM. The PAM sequence varies by the species of the bacteria from which Cas9 was derived. The most widely used CRISPR system is derived from S. pyogenes and the PAM sequence is NGG located on the immediate 3′ end of the sgRNA recognition sequence. The PAM sequences of CRISPR systems from exemplary bacterial species include: Streptococcus pyogenes (NGG), Neisseria meningitidis (NNNNGATT), Streptococcus thermophilus (NNAGAA) and Treponema denticola (NAAAAC).

gRNA(s) used in the present disclosure can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In one embodiment, gRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

To facilitate gRNA design, many computational tools have been developed (See Prykhozhij et al. 2015 PLoS ONE 10(3):; Zhu et al. 2014 PLoS ONE 9(9); Xiao et al. 2014 Bioinformatics. Jan. 21 (2014)); Heigwer et al. 2014 Nat Methods 11(2):122-123). Methods and tools for guide RNA design are discussed by Zhu 2015 Frontiers in Biology 10(4):289-296, which is incorporated by reference herein. Additionally, there is a publicly available software tool that can be used to facilitate the design of gRNA(s) (http://www.genscript.com/gRNA-design-tool.html).

### Human Immune System (HIS) Mice

The availability of highly immunodeficient, NOD-scid-common gamma chain deficient (NSG) mice, that lack murine T, B and NK cells, has greatly enhanced the ability to generate human immune system (HIS) mice. One of the key requirements for generating HIS mice with optimal immune function is the availability of human thymus tissue. Fetal human thymus tissue supports robust human thymopoiesis from injected fetal or adult CD34+ cells, which maintain a steady supply of T cell progenitors to the thymus and in the bone marrow generate B cells, DCs and monocytes that populate the periphery and serve as antigen-presenting cells (APCs) for the T cells developing in the human fetal thymus graft (Lan et al. 2004; Lan et al. 2006; Melkus et al. 2006). T cells developing de novo in the human thymus graft are tolerant of the murine host, presumably due to deletion by murine APCs that are detectable in these grafts (Kalscheuer et al. 1999). While the native murine thymus is capable of generating human T cells at a low level, the abnormal structure of the murine thymus results in a failure of normal negative selection (Khosravi Maharlooei, et al. 2019). This, combined with slow peripheral T cell reconstitution and consequently high levels of lymphopenia induced proliferation (LIP), result in a severe autoimmune syndrome that can be prevented by native mouse thymectomy (Khosravi Maharlooei, et al. 2019). In contrast, the implantation of human fetal thymus tissue in HIS mice receiving CD34+ hematopoietic stem/progenitor cells (HSPCs) results in a human thymus with normal structure, including readily discernable cortex, medulla and Hassal's corpuscles. This human thymus achieves relatively rapid reconstitution of naïve human T cells in the periphery, with markedly reduced LIP and less autoimmunity compared to that observed for T cells developing in the native NSG mouse thymus.

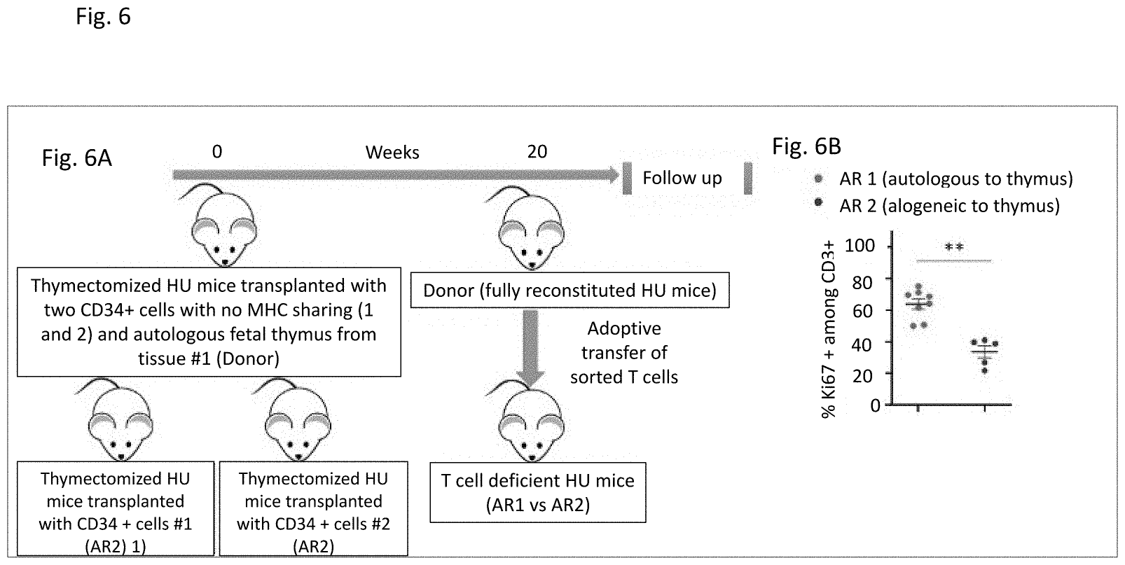

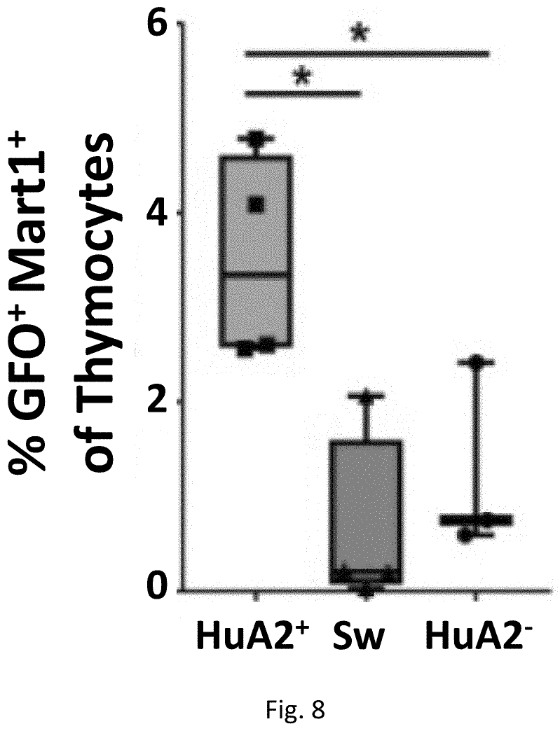

In view of problems with the availability and use of human fetal tissue, it is desirable to identify another source of thymic tissue that could function similarly to that from human fetuses. The inventors have previously shown that robust human thymopoiesis occurs in porcine thymus grafts implanted in immunodeficient mice that receive human HSPCs (Nikolic et al. 1999; Shimizu et al. 2008; Kalscheuer et al. 2014). The use of fetal pig thymus tissue provides an alternative to human fetal thymus tissue that generates normal, functional human T cells, including Tregs, with a diverse TCR repertoire. However, the absence of HLA molecules on porcine thymic epithelial cells (TECs) may limit the selection of human T cells that mediate optimal HLA-restricted immune function in the periphery, as indicated by responses to immunization and the demonstrated failure of pig thymus to positively select thymocytes expressing an HLA restricted transgenic TCR20 (FIGS. 6 and 8). Furthermore, pig thymi may be limited in the ability to positively select HLA-restricted Tregs that recognize human tissue-restricted antigens (TRAs) produced by TECs, and in the negative selection of effector T cells that recognize these TRA/HLA complexes. Peripheral human T cells that were generated in a pig compared to a human fetal thymus show subtle impairments in HLA-restricted immune functions and homeostasis and tolerance to tissue-restricted antigens (Kalscheuer et al. 2012). The addition of transgenic HLA molecules to the porcine thymus tissue could overcome most of these limitations.

Shown herein are two improved methods for obtaining an HIS mouse which do not rely upon the use of human fetal tissue.

In one embodiment, the HIS mouse is generated by introducing fetal thymic tissue derived from a swine and human CD34+ cells into the mouse. In some embodiments, the human CD34+ cells are derived from cord blood. In some embodiments, the human CD34+ cells are derived from adult tissue. In some embodiments, the adult tissue is bone marrow. In some embodiments, the CD34+ cells are derived from mobilized peripheral blood hematopoietic stem cells.

In a further embodiment, the HIS mouse is generated by introducing fetal thymic tissue derived from a transgenic swine described herein.

In some embodiments, the mouse is thymectomized prior to the introduction of the thymic tissue as recently described (Khosravi Maharlooei et al. 2019). In some embodiments, the mouse is also irradiated. In some embodiments, the mouse is a NOD scid common γ chain knockout (NSG) mouse.

The swine fetal thymus can be implanted under the kidney capsule of the mice. If the mice are being injected with the human cord-blood derived CD34+ cells, they can be injected before, after or simultaneously with the implantation of the thymus.

The HIS mouse model can be extensively applied to research areas where T cells play an important role. These areas will include, but not be limited to:

HIV infection and other infections. This model has been used to demonstrate that pig thymus confers resistance to HIV infection compared to human fetal thymus tissue (Hongo et al. 2007).

Treg biology, including development in thymus, trafficking and homeostasis in peripheral tissues. This model has been used to demonstrate excellent Treg development and function when they are generated in a pig thymus, but with subtle phenotypic differences due to altered peripheral homeostasis, which is expected to be corrected by the addition of HLA molecules to the thymic tissue. In addition, this model will be useful for studying Treg therapy as it allows determination of the distribution, survival and activities (e.g., suppressing graft rejection) of ex vivo expanded Tregs following infusion.

Transplantation immunology. HIS mice constructed with human or pig fetal thymic tissue and human fetal or adult CD34+ cells have been shown to be capable of rejecting human and pig skin and islet allografts and xenografts (Lan, et al. 2004; Shimizu, et al. 2008; Zhao, et al. 1997; Zhao, et al. 1998), while those generated with pig fetal thymic tissue specifically accept skin grafts sharing the SLA of the thymus donor (Kalscheuer et al, 2014). The mice generated as described herein can be used to reject allogeneic human skin grafts. These data indicate that this model will be valuable for transplantation immunology and pre-clinical studies to investigate approaches to inducing tolerance to allografts and xenografts. The model will also optimize the mixed chimerism and porcine thymic transplantation approaches to xenograft tolerance that are currently being explored.

Autoimmunity. With the transduction of CD34+ cells with a TCR recognizing an islet autoantigen, this model will facilitate the study of development of autoreactive T cells in the thymus and how tolerance to autoantigens is regulated in both the thymus and periphery. TCRs specific for additional autoantigens can readily be studies in this well-defined model with highly reproducible thymic HLA genotypes.

Infections such as COVID-19. There is a dire need for models that include human immune systems to examine their impact on COVID-19 pathology. The unavailability of human fetal tissue presents a major challenge to such research. This challenge could be met by using HLA-transgenic fetal pig thymus tissue instead of human fetal thymus.

The use of the transgenic swine to generate the HIS mouse can result in a better model than the HIS mouse generated using fetal human thymus because the background MHC (SLA) and HLA transgenes are the same for each donor and the pigs are overall quite inbred. One of the big challenges in using human fetal tissue is that the HLA and entire genetic background is different from donor to donor and this introduces variables that impede the reproducibility of HIS mouse studies.

### EXAMPLES

This invention will be better understood from the Experimental Details, which follow. However, one skilled in the art will readily appreciate that the specific methods and results discussed are merely illustrative of the invention as described more fully in the claims that follow thereafter.

### Example 1—Genetic Modifications in Pig Using CRISPR-Assisted Homologous Recombination

Two pig genetic modifications were made to illustrate that CRISPR-assisted homologous recombination enables genetic modification in pigs when combined with appropriate selection strategies for properly targeted cells.

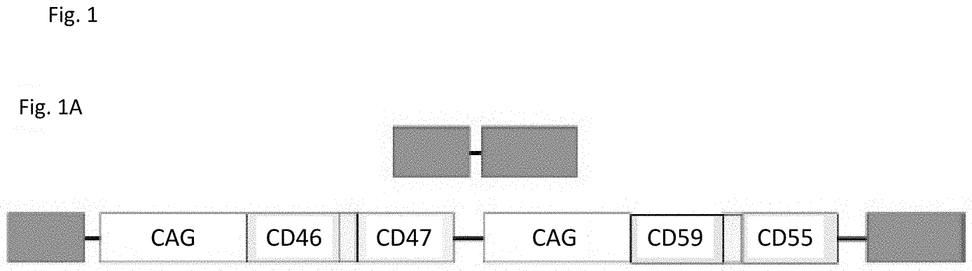

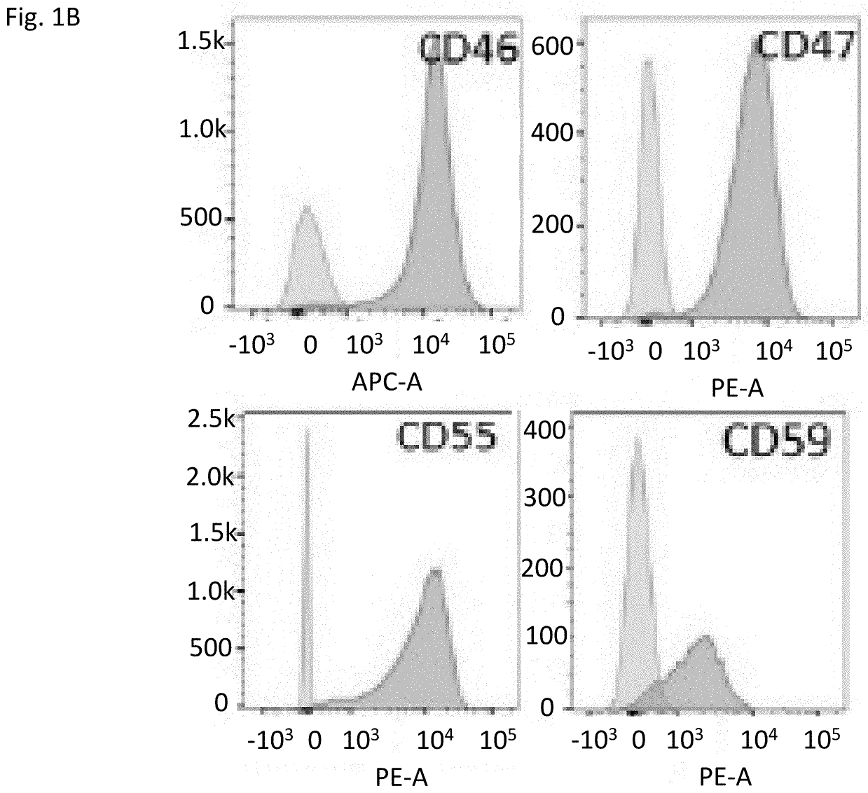

In the first modification, coding sequences for 4 human genes were introduced into the GGTA1 locus of the Sachs miniature swine using CRISP-assisted homologous recombination (FIG. 1). In this case, targeting into the GGTA1 locus provided a “safe harbor” for expression of the transgenes, as this genomic region is not subject to stringent temporal or lineage dependent transcriptional repression. The four transgenes were expressed from the ubiquitous CAG promoter in two groups using 2A self-splicing elements. Non-clonal selection of properly targeted cells was in this case straightforward, as expression of the transgenes could be used as a positive marker and because the vector was transfected into cells heterozygous for a null GGTA1 allele, loss of GGTA1 expression. The rapid, population based selection of cells resulted in a somatic cell nuclear transfer (SCNT) donor population efficient in production of cloned fetuses and piglets.

The second modification was serially introduced into fibroblasts from cloned fetuses carrying the first modifications and was considerably more complex. In this case, coding sequences for both chains of the human IL-3 receptor under the control of the native IL-3 receptor alpha chain promoter were to be introduced in order to achieve appropriate lineage and temporal specificity of human IL3R expression. The major obstacle to targeted cell selection in this case is the lack of IL3R expression in fibroblasts required for SCNT cloning. Additionally, since destructive loss of endogenous ILR3 expression via targeted integration of indel generation is expected to be a highly deleterious if not lethal event, genetic modification to 1 allele of the native ILRa locus was to be limited. From a cloning perspective, the desire was to obtain a non-clonal donor cell population with sufficient enrichment for properly targeted cells in as few population doublings as possible.

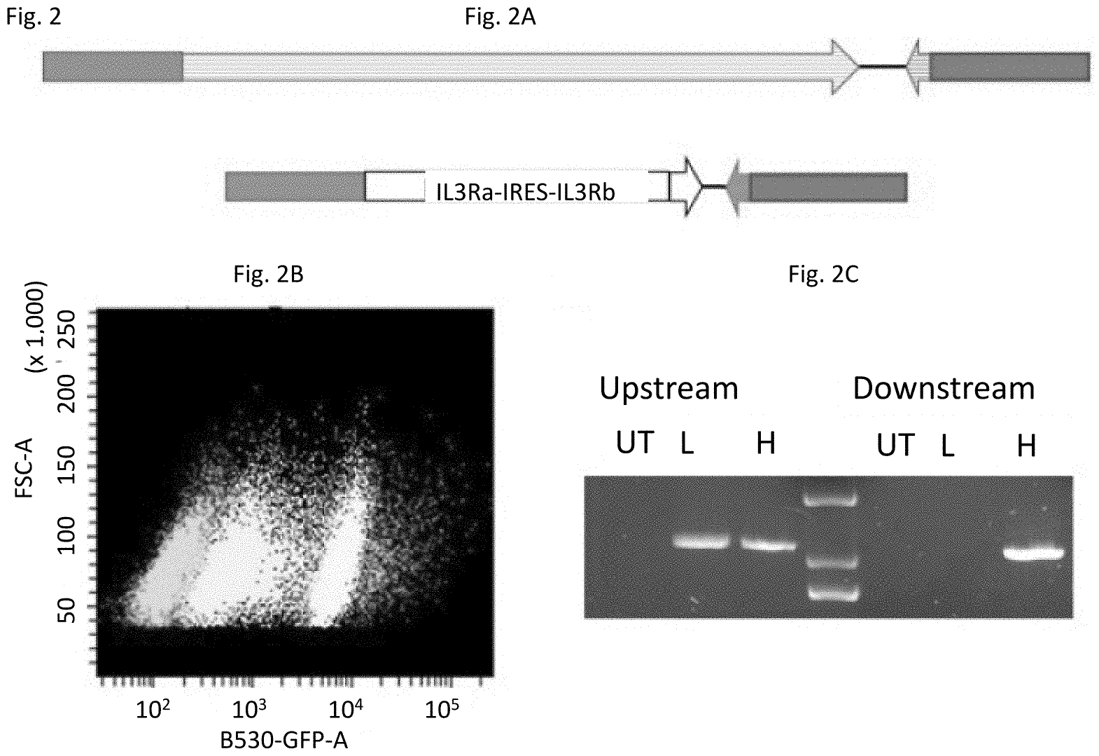

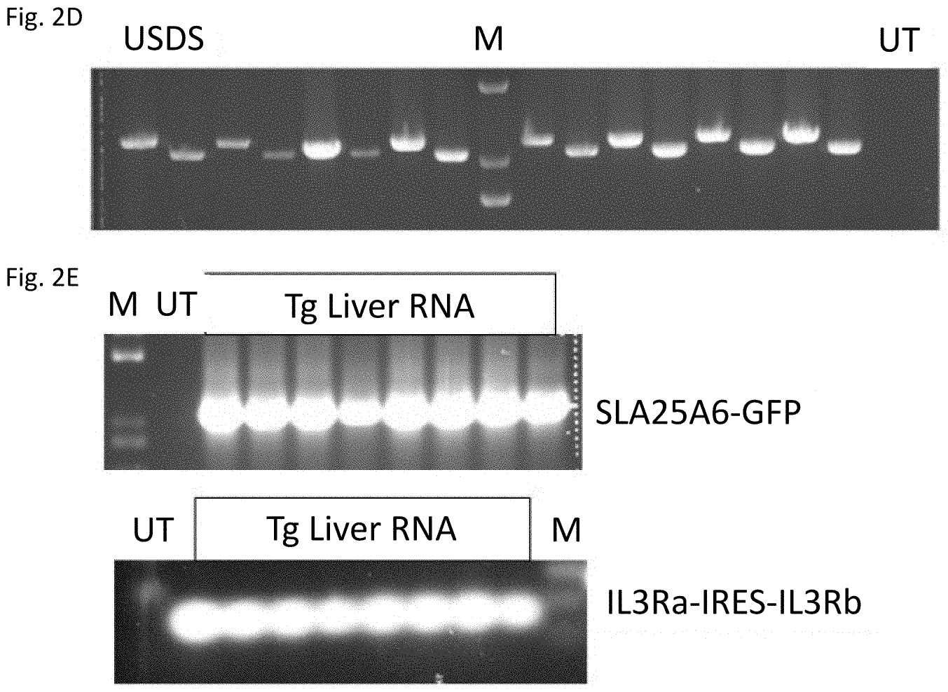

The strategy and results from this study are shown in FIG. 2.

The desire for a highly enriched SCNT donor cell population with minimal doublings indicated that a vector without a selection marker promoter be utilized. Since IL3Ra is not expressed in fibroblasts, it was decided to see if ubiquitous expression of a nearby gene (SLC25A6, a mitochondrial nucleotide transporter) could be utilized as a marker of proper targeting. Although tagging the SLC25A6 transcriptional unit using GFP coding sequences linked via a 2A self cleaving peptide provided a solid selection strategy, it was unclear whether such a complex modification (substitution of >15 kbp of genomic sequence with >7 kbp of vector sequence) could be done with sufficient efficiency for donor cell selection.

A CRISPR guide RNA expected to cleave 1 allele of the IL3Ra gene in the previously modified fetal cells was selected and tested along with the illustrated vector. In preliminary transfections, it was found that use of paired guide RNAs in combination with a “nickase” form of Cas9 generated populations that included fairly discrete GFP high and low subpopulations. Flow analysis of the population generated with 1 such combination is shown in FIG. 2B. PCR analysis indicated that cells in the sorted GFP high subpopulation contained cells with proper integration of both ends of the vector (FIG. 2C). Cells in this population were used in SCNT at approximately 24 doublings (well before mean clonal senescence at 32 doublings), resulting in the generation of 8 viable fetuses from 3 embryo recipient gilts. Genomic and RT-PCR analysis showed that all 8 fetuses carried the intended genetic modification (FIGS. 2D and 2E). Additional pregnancies using this donor cell population were continued to term and live births expressing the relevant transgenes were obtained.

Together, the modifications described here demonstrated that multicistronic targeted modifications can be serially introduced into pigs using non-clonal donor cell selection strategies to rapidly generate pigs carrying multiple genetic modifications

### Example 2—HLA-A2 Transgenesis: Production and Genotypic/Phenotypic Evaluation of d40 Transgenic Pig Fetuses

### Starting Material

Fibroblasts from GGTA1 null, SLA haplotype h homozygous Sachs Miniature Swine (SLA-1*02:01, SLA-2*02:01, SLA-3 null, SLA-DRA*01:01:02, SLA-DRB*02:01, SLA-DQA*02:02:01, SLADQB* 04:01:01) is used as the starting material for genetic modification. Cells from this line have cloned well in previous transgenic projects and a large breeding population is maintained by CCTI for xenotransplantation studies, facilitating expansion of HLA transgenics for supply of thymic tissue to the research community. Due to the partially inbred nature of these animals, offspring will have a high degree of genetic similarity.

### Overall Strategy

All transgenic modifications are made by targeted insertion behind native SLA promoters. This will ensure appropriate lineage and temporal expression patterns. This also avoids potential problems associated with inappropriate placental HLA expression during development. Both chains of the transgenic molecules are simultaneously introduced. Serial modifications are employed at the fetal stage to rapidly generate first HLA-A2 transgenic thymic material and then HLA-A2/HLA-DQ8 transgenic thymic material.

Promoter-less gene targeting vectors are used to introduce both the HLA modifications, allowing selection of non-clonal cell populations highly enriched for properly targeted cells with a minimal number of cell divisions prior to use in somatic cell nuclear transfer (SCNT). While this is a similar approach as used in Example 1 for promoter targeted modification with the IL3 receptor chains, the vector design process is considerably simplified as both Class I and Class II molecules are normally or inducibly expressed in fibroblasts required for SCNT cloning.

### Production of the d40 Cloned Transgenic Fetuses

Coding sequences for HLA-A2 are introduced behind either SLA-1 or SLA-2 Class I promoters. These loci are interchangeable with respect to the intended modification and the choice of one will be determined by intron 1 sequencing of both and evaluation for optimal CRISPR guide RNA sites.

HLA-A2 is expressed as a fusion of human beta-2 microglobulin (B2M) with the HLA-A2 alpha chain. Transgenic expression of such a fusion has previously been described in mice (Kotsiou et al. 2011; Pascolo et al. 1997) and its use here ensures that heterotypic interactions between HLA-A2 and pig B2m will not interfere with HLA-A2 surface expression.

CRISPR/Cas9-assisted homologous recombination is used to target the fusion cassette. The HLA-A2 targeting is limited to one allele of the SLA I gene and that the other allele will may be rendered null; mutation of the second allele would be without immune consequence in the pig and may increase HLA-A2 expression through decreased expression of endogenous Class I alpha chain.

### Vector Construction for Integration of HLA-A2

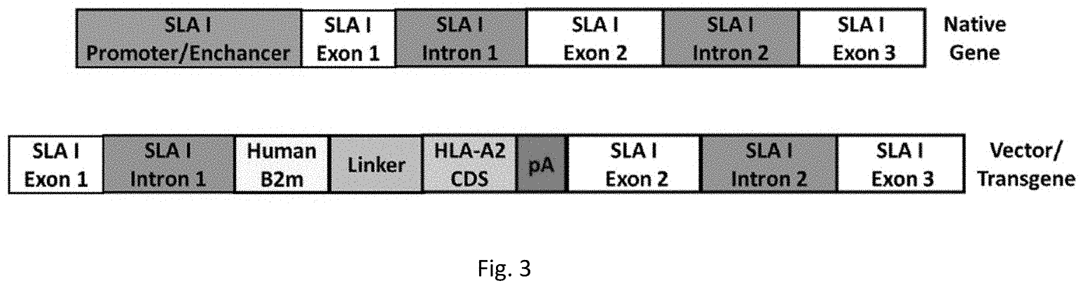

The targeting vector for integration of HLA-A2 is diagrammed in FIG. 3. Homologous recombination between vector homology arms identical in sequence to those in the native gene (white and blue segments) results in the introduction of the human B2M-HLA-A2 cassette at the intron 1/exon 2 junction. The mature form of human B2M is introduced here, with the signal peptide provided by exon 1; since the signal peptide ends 1 bp from the splice site, the fusion protein is made without alteration of the B2M protein sequence. Paired CRISPR guide RNAs are selected at appropriate sequence sites near the end of intron 1 and beginning of exon 2 and incorporated into plasmids expressing Cas9 nickase activity.

### Selection of Modified Fibroblasts for SCNT

Targeting and CRISPR/Cas9 guide plasmids are nucleofected into fibroblasts and subjected to first round selection 3-5 days later. Selection is by flow sorting of cells stained with an HLA-A2-specific antibody (clone BB7.2, Biolegend). A preliminary, single sort analysis is performed with chosen guide pairs to determine the pair yielding the highest targeting rate based on HLA-A2 expression. For SCNT donor cell selection, two rounds of similar selection is employed for maximal enrichment of expressing cells. This population is then subjected to genomic and RT-PCR analyses to confirm the expected structure and RNA level expression of the transgenic locus and to determine if the second SLA locus has been altered in the process.

### Production and Characterization of d40 Transgenic Fetuses

Selected SCNT donor cells are used for nuclear transfer/embryo transfer, with resulting fetuses harvested at approximately 40 days gestation. A two-stage cloning process is employed in all of pig engineering projects. Harvest at 40 days gestation allows confirmation of genetic structure, and often transgene expression, at a clonal level prior to committing to a line for further clone production. Additionally, minimally cultured cells from early fetuses tend to have a much higher cloning rate than those following an extended in vitro selection process. Finally, it allows “renewal” of a line with respect to in vitro lifespan, essential for additional genetic modification (e.g., serial introduction of HLADQ8).

For characterization of HLA-A2 transgenic fetuses, genomic PCR is used to confirm expected integration site structure, RT-PCR to confirm proper RNA expression and flow cytometric analysis to confirm cell surface expression.

### Example 3—HLA-A2/HLA-DQ8 Transgenesis: Production and Genotypic/Phenotypic Evaluation of d40 Transgenic Pig Fetuses

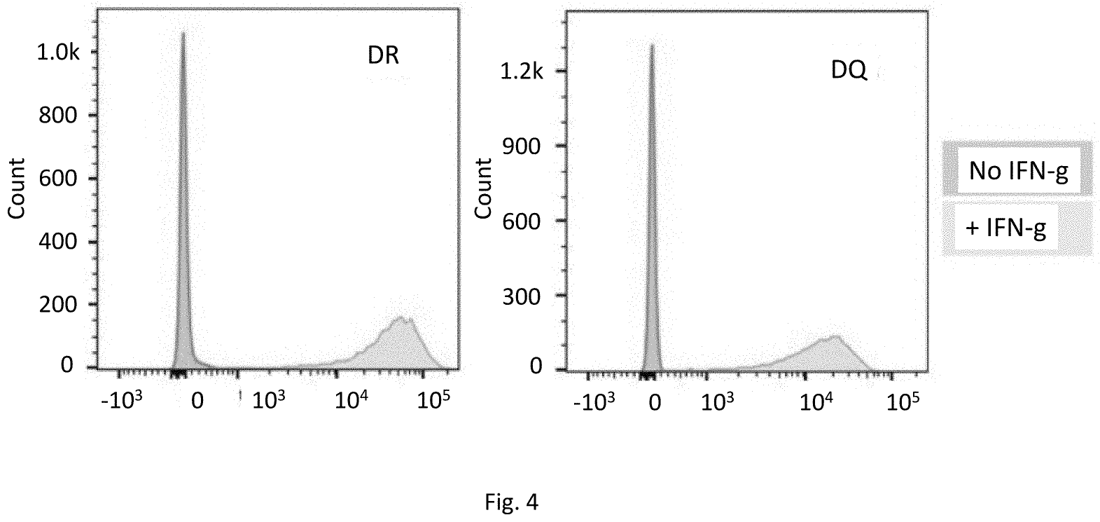

A transgenic pig (HLA-A2,/HLA-DQ8) is produced using a similar overall strategy and targeting expression with a promoterless vector to a native promoter with cell selection based on HLA-DQ8 expression described in Example 2. In contrast to SLA Class I, SLA Class II is not normally expressed on fibroblasts. To determine if Class II expression could be induced in fetal fibroblasts with interferon gamma, as is observed in human and mouse fibroblasts, primary fetal fibroblasts were exposed to porcine IFN-g (80 ng/ml) and then porcine DR and DQ pan-allelic surface expression was observed by flow cytometry. Surface expression of both DR and DQ was found to be strongly induced in nearly all cells following 6 days of treatment with IFN-g (FIG. 4), with the majority of cells strongly expressing both after 3 days of induction. Importantly, such treatment appeared to have no effect on the morphology or growth of these cells. Induced expression of Class II is therefore a viable means of selecting for native Class II promoter expression of transgenic HLA-DQ8 in cells required for SCNT cloning.

Proper Class II expression is dependent on the function of accessory molecules, including CD74 and, in humans, HLA-DM. Expression of HLA-DQ8 in transgenic mice makes it likely that pigs also have all the appropriate activities for HLA-DQ8 expression as well (Cheng et al. 1996). The murine study indicated that expression of endogenous MHC-II molecules can limit exogenous MHC-II expression, presumably through competition. HLA-DQ8 expression is targeted to the native SLA-DQA locus. The targeting event will in itself result in loss of function of one SLA-DQA allele. Due to the nature of CRISPR-mediated modifications, the indel associated loss of function will occur at the non-targeted allele as well in a large proportion of cells.

### Vector Construction

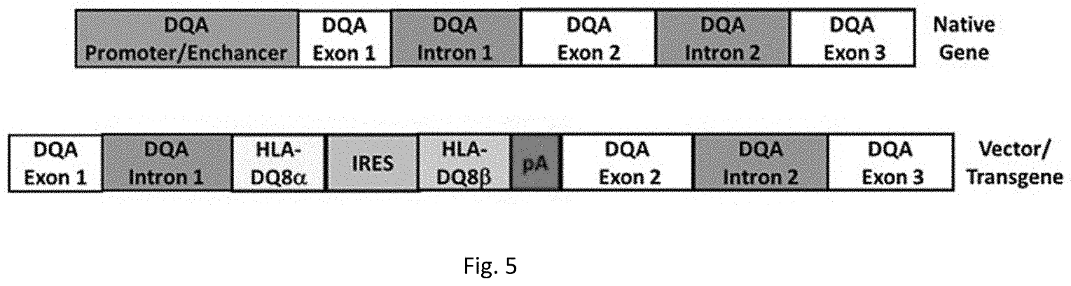

The targeting vector for integration of HLA-DQ8 is diagrammed in FIG. 5.

As for HLA-A2 transgenesis, both alpha and beta chains is introduced in a single transgenic step. For DQ8, coding sequences for the two chains are linked with a high efficiency IRES element that has been successfully utilized in other bicistronic expression vectors. An IRES linkage is preferred here to a self-splicing element, as the functional consequences of addition of amino acids to the HLA-DQ alpha chain are unknown. Also like the HLA-A2 addition, exon 1 of the native locus is used to supply the leader sequence for HLA-DQ8, resulting in a single amino acid addition to the N-terminus.

### Selection of Modified Fibroblasts for SCNT

HLA-A2 transgenic d40 fetal cells produced in Example 2 is the starting material for introduction of the HLA-DQ8 modification. Preliminary and SCNT donor cell transfection is performed as described in Example 2. Numerous anti-pan haplotype human DQ antibodies are commercially available. Selection candidates are screened first on IFN-g-induced pig fibroblasts to identify candidates which do not bind pig DQ dimers. A second screen is then performed on these candidates using IFNg-induced pig fibroblasts transfected separately with expression constructs for HLA-DQA*03:01 and HLADQB1*03:02 to eliminate any antibodies that recognize cross-species dimers. Cell selection with the candidate(s) which meet these criteria is the performed as described in Example 2. The flow sorted population is subjected to genomic and RT-PCR analyses to confirm the expected structure and RNA expression of the transgenic locus, also as in Example 2.

### Production and Characterization of d40 Transgenic Fetuses:

Genomic and RNA analyses will be conducted as described for the HLA-A2 modification in Example 2.

### Example 4—Production of d56-70 Thymic Tissue Expressing HLA-A2 and HLA-A2/HLA-DQ8

Genotypically and phenotypically confirmed early fetal cell lines produced from Examples 2 and 3 are sent to a facility with laboratories for cell culture, oocyte maturation and embryo reconstruction as well as surgical facilities from embryo transfer and deliver of fetuses and piglets. SCNT cloning to produce day 56-70 fetuses is performed. Thymic isolation is performed by methods known in the art after conformation genotyping and phenotyping of the fetuses.

### Example 5—Breeding of HLA-A2/HLA-DQ8 Transgenic Founder Boars

SCNT for founder boars utilizes d40 fetal cells of confirmed genotype/phenotype produced in Examples 2 and 3. Transgenic piglets are reared to shipping age (8-16 weeks) and sent to a state of the art farming facility for large animal breeding, housing and procedures for further husbandry.

### Example 6—Importance of HLA Sharing Between the Thymus and Peripheral APCs for Human T Cell Homeostasis in HIS Mice

### Methods

6-8 week-old female NOD scid common γ chain knockout (NSG) mice, purchased from the Jackson Laboratories, were thymectomized as previously described (Khosravi Maharlooei et al. 2019). Two weeks later, these mice received sublethal total body irradiation (1 Gy) followed by surgical implantation of a 1 mm³ fetal pig or human thymic tissue fragment under the kidney capsule.

Mixed chimeric donor HIS mice were then generated by transplantation of two sets of allogeneic CD34+ cells with no HLA sharing (#1 and #2) and autologous fetal thymus from donor #1 to thymectomized NSG mice. Two groups of adoptive recipient (AR) mice were generated by injection of CD34+ cells #1 or #2 to thymectomized NSG mice (no thymus). At 20 weeks post transplantation, T cells from mixed chimeras were injected i.v. to AR1 and AR2 mice. See FIG. 6A.

### Results

At day 10 post adoptive transfer, the proportion of proliferating (Ki67+) T cells was significantly greater in AR1 mice, in which the APCs were HLA-autologous to the donor thymus that selected the T cells, than in AR2 mice bearing only allogeneic HLA. See FIG. 6B.

These studies demonstrate that thymic HLA on peripheral APCs is needed to support maximal lymphopenia-driven expansion of peripheral human T cells, highlighting the importance of studies to provide human thymic epithelial cells or HLA molecules in a swine thymus to achieve normal immune homeostasis.

### Example 7— Comparison of Human Immune Reconstitution in HIS Mice Methods

Humanized mice were generated by the implantation of pig fetal thymi under the kidney capsule of thymectomized irradiated NOD scid common γ chain knockout (NSG) mice as described in Example 6.

These mice were then injected with human cord blood-derived CD34+ cells. Two batches humanized mice were generated using the same fetal pig thymus and different cord blood CD34+ cells. CD34+ cells will be isolated by using the human CD34 microbead kit (Miltenyi Biotech). Anti-CD2mAb LoCD2b (400 μg/mouse) was injected intraperitoneally once a week for 2 weeks (Days 0, 7 and 14) for depletion of residual T cells in the CD34+ cell inoculum and of residual thymocytes released from human fetal thymic tissue to prevent rejection of pig thymus tissue and/or injected allogeneic human cord blood CD34+ cells by pre-existing human thymocytes from the graft.

Reconstitution of humanized mice generated with human fetal thymic tissue and autologous fetal liver-derived CD34+ cells in a different experiment was included for comparison.

Starting at week 4, peripheral blood of the mice was obtained and blood concentrations of human CD3 cells measured.

At week 15, flow cytometric analysis of peripheral blood was performed to determine numbers of T, B and myeloid cell populations, including CD4 and CD8 T cells, naïve and memory CD4 and CD8 T cells, regulatory T cells (Tregs) and T follicular helper (Tfh) cells; B cell subsets, monocytes and dendritic cells (DCs), including classical DCs (cDC1s and cDC2s) and plasmacytoid DCs (pDCs).

### Results

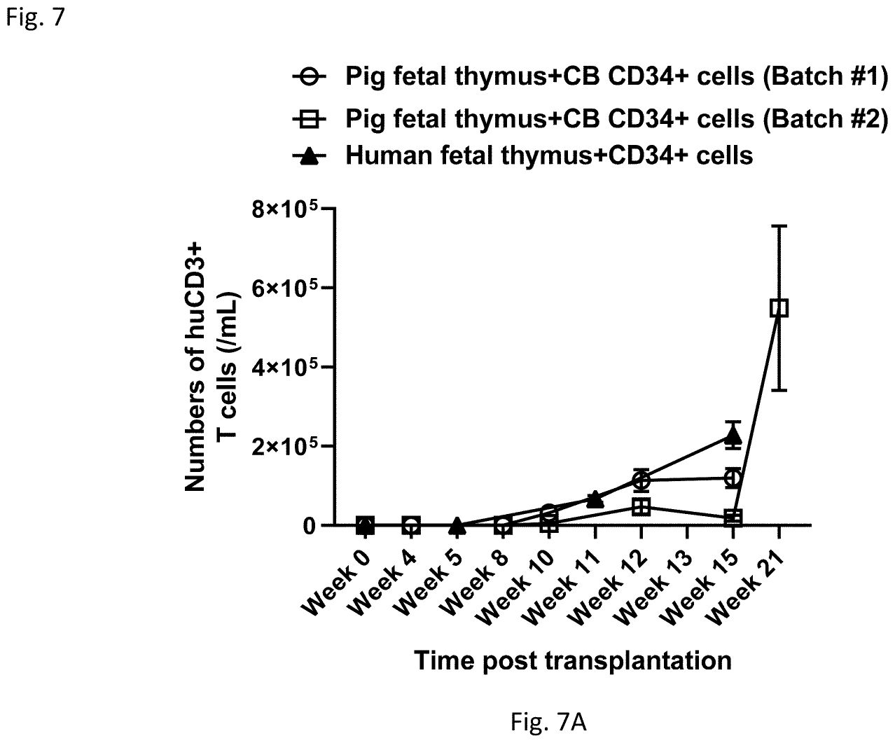

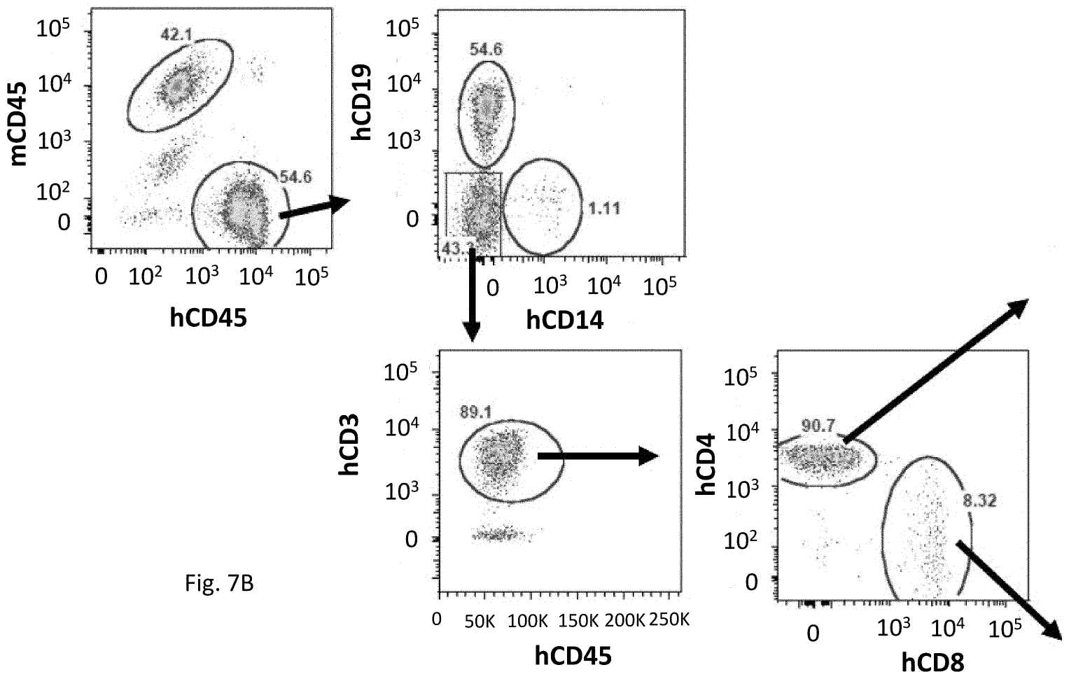

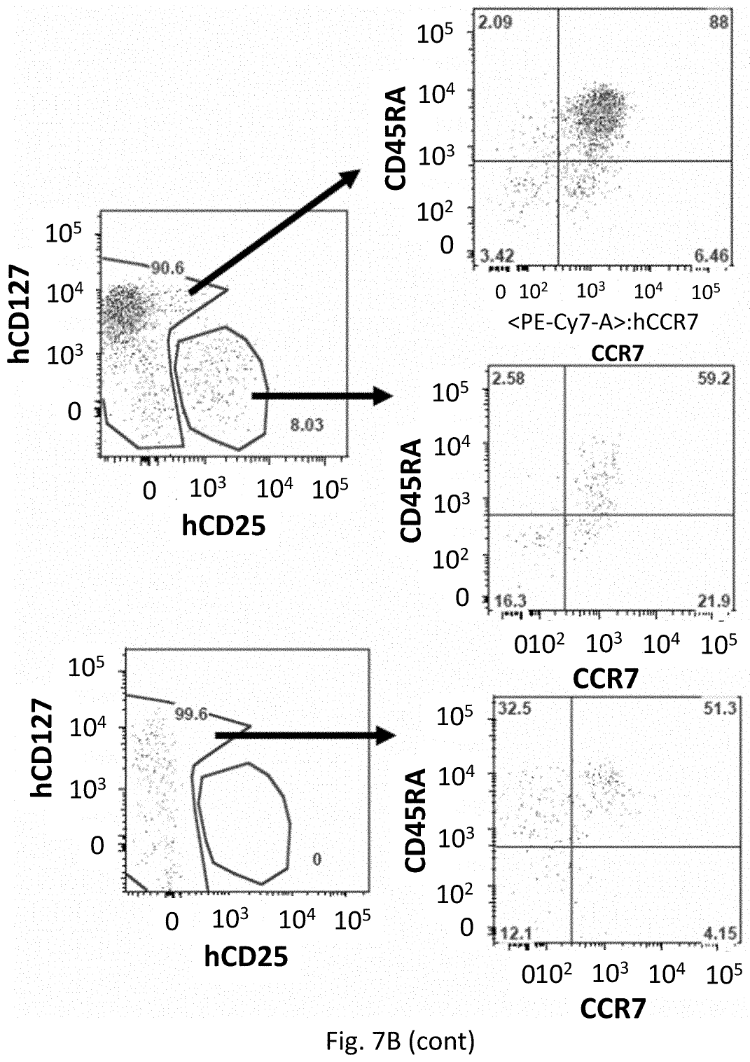

As shown in FIG. 7A, based on human cells in the peripheral blood of the mice, human T cell reconstitution was comparable in the two batches of mice generated with pig fetal thymus and human CD34+ cells to those generated with human fetal thymus.

As shown in FIG. 7B, a high percentage of naïve T cells in CD4 and CD8 subsets was detected. Generation of CD4+CD25^{high} CD127^{low} regulatory T cells was also demonstrated.

### Example 8—Continued Monitoring and Analysis of HIS Mice

The mice generated in Example 7 are further monitored as follows.

Monitor and compare plasma immunoglobulin levels (IgM and IgG) by ELISA every 4 weeks following transplantation.

14-16 weeks post-transplantation, when HIS mice are expected to fully be reconstituted by human cells, half of the animals in each group are euthanized and the size, structure, cellularity and cell populations within peripheral blood, lymph nodes, spleen and thymus are compared of all groups. Flow cytometry panels to study immune cell populations are those shown in Table 1. A small piece of each lymphoid tissue, including spleen, lymph node and thymus, is used for histological studies to compare the structures of these tissues. Serum immunoglobulin levels (IgM and IgG) are measured by ELISA in all HIS mice. In addition, the function of human T cells in the periphery of each group of mice is compared using in vitro assays of proliferation, cytokine production and cytotoxicity in response to pan-TCR stimulation (anti-CD3/CD28 beads), alloantigen stimulation, xenoantigen stimulation and tetanus toxoid neoantigen stimulation. Proliferation is determined by CFSE cellular dye dilution. Production of cytokines, including IL-2 and IFN-γ, is assayed by intracellular staining. For alloantigen and xenoantigen stimulation, allogeneic human PBMCs and 3rd party pig PBMCs are used as stimulators. Isolated splenic T cells from HIS mice are labeled with CFSE and co-cultured with irradiated stimulators at a ratio of 1:1 for 6 days. CFSE dilution of human CD4 and CD8 T cells is determined by flow cytometry. For tetanus toxoid neoantigen stimulation, DCs are generated using the cord blood or fetal liver-derived CD34+ cells that are used for generation of HIS mice. CD34+ cells are cultured with human cytokines, including stem cell factor, GM-CSF and IL-4 for 13 days for differentiation into dendritic cells. CD34-derived DCs are pulsed with tetanus toxoid neoantigen and then matured by TNF-α and PGE2 followed by coculture with CFSE-labeled isolated splenic T cells for 7 days. Proliferated T cells are determined by flow cytometry. Monocytes will be stimulated with LPS and production of TNF-α, IL-6 and IL-10 in supernatant is determined by ELISA.

The remaining HIS mice are monitored up to 30 weeks to observe the persistence of reconstitution of each lineage and to observe for the emergence of graft-vs-host/autoimmune disease. Mice are bled every 4 weeks to determine human cell engraftment. Starting from 20 weeks post-transplantation, mice are scored for graft-vs-host disease/autoimmunity twice per week until week 30 using the scoring system shown below. All analyses will be the same as those described above.

### Scoring System:

Weight loss (%): <10%, 0; <10-15%, 1; <15-20%, 2; >20%, 3
Posture: Normal, 0; Mildly hunched at rest, 1; Moderately hunched, able to ambulate normally, 2; Severe hunching, impairs movement and gait, 3
Hair coat: Normal, 0; Mild ruffling, 1; Moderate ruffling, 2; Severe ruffling, Porphyrin staining of face or forelimbs, 3
Activity: Normal, 0; Mild to moderately decreased, 1; Active only to eat, drink or when stimulated, 2; difficulty rising, unable to move when stimulated, 3

Animals with any signs of GVHD (score greater than 2) are monitored daily with weight checked every other day. Animals with a total score of 6 or higher are monitored and weighed daily. Animals with a total score of 9 or higher or a score of 3 in any one category are euthanized.

These studies compare human reconstitution following transplantation of fetal pig thymus and cord blood derived CD34+ cells versus that achieved with fetal human thymus and fetal CD34+ cells. The results show that the HIS mice generated with fetal pig thymus and cord blood derived CD34+ cells have similar human reconstitution to those HIS mice generated with fetal human thymus and fetal CD34+ cells. Once human cell reconstitution is confirmed in peripheral blood (about 4 months following transplantation), studies to investigate the in vivo immune function of these mice by determining thymic selection of transgenic human T cell receptors (TCRs) with defined restriction and rejection of human allogeneic skin grafts, as described below, are initiated.

TABLE 1
Antibody panels to study subsets of T, B and DCs

| T cell panel | B cell panel | DC panel |
|---|---|---|
| ICOS-PE-Cy7 | CD14-APC-Cy7 | CD14-PE |
| CD45RA-AF488 | CD38-PE-Cy7 | HLA-DR-FITC |
| CCR7-PE | CD27-BV711 | CD11c-PE-Cy7 |
| BLC6-PE-CF594 | IgM-PE-CF594 | CD1C-AF700 |
| PD-1-PERCP-Cy5.5 | CD21-PERCP-Cy5.5 | CD3&CD19- PERCP- |
| | | Cy5.5 |
| IL-10-APC | CD3-PE | CD123-BV711 |
| IL-21-AF647 | CD19-BV650 | CD141-BV605 |
| Mouse CD45-APC- | Mouse CD45-BV450 | Mouse CD45-APC-Cy7 |
| Cy7 | | |
| CXCR5-BV421 | CD20-APC | CD303-APC |
| CTLA-4-BV605 | CD138-AF700 | Human CD45-V500 |
| CD8BV650 | IgD-BV605 | |
| CD25-BV711 | CD24-BUV395 | |
| CD3-BV785 | Human CD45-FITC | |
| Human | CD45-Qdot800 DAPI | |
| FOXP3-AF700 | | |
| CXCR3-BB700 | | |
| VD4-V500 | | |
| CD127-BV570 | | |
| Viability-NIIR | | |

### Example 9—Comparison of Selection of an HLA-A2 Restricted TCR in HIS Mice

The selection of an HLA-A2-restricted TCR in SLA-defined fetal thymic tissue vs HLA-A2+ fetal human thymus tissue in thymectomized NSG mice reconstituted from cord blood CD34+ cells is compared. Using lentiviral transduction of human CD34+ cells in HU/HU mice, it has been established that the human HLA-A2-restricted TCR MART1 was positively selected in an HLA-A2+ human thymus but not in an SLAkm porcine thymus (FIG. 8). This study shows that this TCR also fails to be positively selected in a homozygous SLAhh fetal pig thymus, since this is the pig SLA that is used for introduction of the HLA transgenes in the transgenic pigs of Examples 2 and 3.

Three groups of mice are generated using fetal pig thymus (SLAhh) or fetal human thymus and MART-1-TCR-transduced fetal liver or cord blood-derived CD34+ cells (Table 2) as described generally in Example 7. For transduction of CD34+ cells, human fetal liver or cord blood CD34+ cells are pre-stimulated in retronectin-coated plates by incubation in Stemline II medium with 10 μg/mL protamine sulfate and 60 ng/mL, 150 ng/mL and 300 ng/mL recombinant human IL-3, Flt3 Ligand, and stem cell factor, respectively, for 3 hours. Cells are transduced overnight at a multiplicity of infection of 30, then harvested and prepared for intratibial injection. A small number of transduced CD34+ cells are cultured in stem cell medium without protamine sulfate for 4 days, then assessed for transduction efficiency by flow cytometry. HLAA2+ fetal liver or cord blood CD34+ cells are used to generate HIS mice, as the presence of HLA-A2+ APCs in the periphery is likely required for optimal homeostasis of human T cells selected by HLA-A2. For HLA typing, DNA is isolated from CD34 negative fetal liver or cord blood cells using the DNeasy Blood & Tissue Kit (Qiagen) following isolation of CD34+ cells from these tissues. Sanger allele-level HLA typing is performed to determine the HLA type of the tissues. While the tissues are being typed, human fetal and cord blood CD34+ cells are frozen.

14-16 weeks post-transplantation, when HIS mice are fully reconstituted by human cells, they are euthanized for analysis. The percentages and absolute numbers of MART-1+ thymocytes among double negative (CD1a+), including CD7+ early thymocytes, double positive, CD4 single positive and CD8 single positive subsets are determined along with markers of selection (CD69, PD1,CCR7). Failure of positive selection of the HLA class I-restricted TCR MART1 in fetal pig thymus is observed.

Fluorochrome-labelled MART1 tetramer is used to identify transgenic T cells and GFP serves as a marker of origin from a transduced HSPC. GFP+ and GFP− thymocytes at each stage of thymic development provides internally-controlled comparisons of the level of selection of transgenic and non-transgenic T cells in each individual mouse. These studies, conducted as the transgenic pigs are being produced (Examples 3 and 4), provide a baseline against which to determine the effect of HLA-A2 transgenes in fetal pig thymus on selection of HLA-A2-restricted human T cells in a pig thymus. The detailed panel is shown in Table 3 below. Analysis will be performed in Aurora Spectral flow cytometry.

TABLE 2
HIS mice made with fetal human and fetal non-human (porcine) thymus
tissues

| Group | | |
|---|---|---|
| cells | HLA-A2+ Thymic tissue | MART-1 TCR-transduced CD34+ |
| 1 | Fetal human thymus HLA-A2+ | Fetal liver derived (autologous) |
| 2 | Fetal pig thymus (SLAhh) | HLA-A2+ Fetal liver derived |
| 3 | Fetal pig thymus (SLAhh) | HLA-A2+ Cord blood derived |

TABLE 3
Panel to study selection of MART-1+ T cells in thymus

| | |
|---|---|
| GFP | GFP |
| Tetramer | APC |
| Mouse CD45 | V450 |
| Human CD45 | QDot800 |
| CD3 | BV786 |
| CD4 | V500 |
| CD8 | BV480 |
| CD69 | BV650 |
| CD1a | PerCP-efluor710 |
| CD5 | BV711 |
| PDI | PE-Dazzle 594 |
| CD34 | BV785 |
| CD38 | PE-Cy7 |
| CD7 | PE-Cy5 |
| CD31 | BV605 |
| CCR7 | BV421 |
| CD45RA | APC-H7 |
| CD25 | AF700 |
| CD127 | BV570 |
| Viability | Zombie NIR Dye |

### Example 10—Comparison of Selection of an HLA-DQ8-Restricted Islet Autoantigen-Specific TCR in HIS Mice

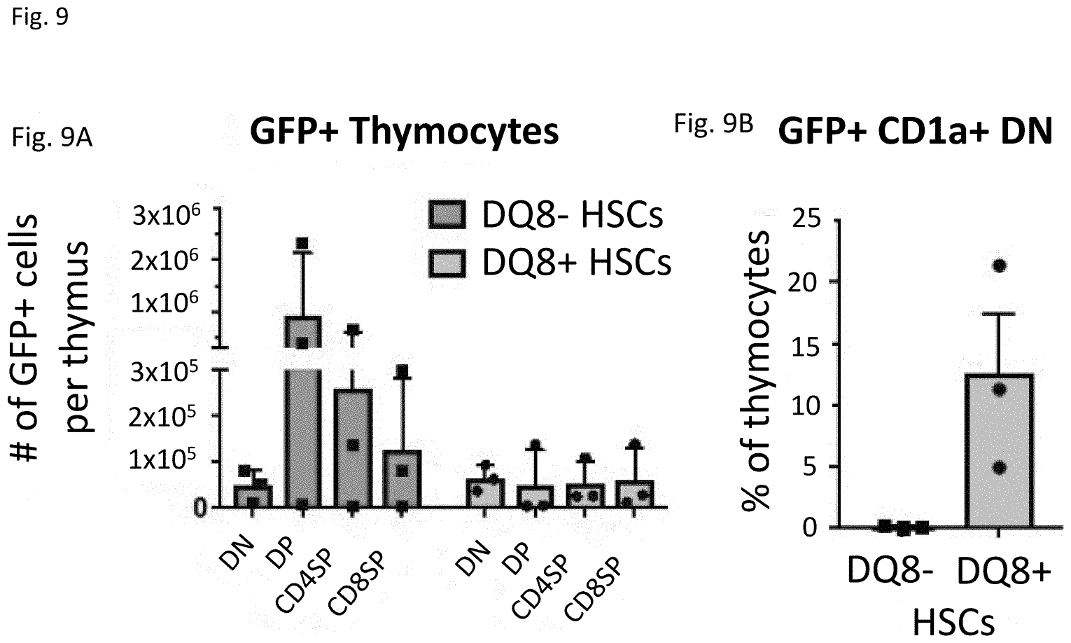

Next the selection of an HLA-DQ8-restricted islet autoantigen-specific TCR, Clone 5, is compared in SLA-defined fetal thymic tissue vs fetal human (bearing the relevant HLA allele for each TCR) in thymectomized NSG mice reconstituted from HLA-DQ8+ cord blood CD34+ cells. Using human fetal thymus tissue, it has been shown that Clone 5 TCR+ T cells are positively selected in an HLADQ8 human fetal thymus and negatively selected if the HSPCs express HLA-DQ8 (FIG. 9). Three groups of HIS mice (Table 4) are generated using fetal pig thymus (SLAhh) or fetal human thymus and Clone 5 TCR-transduced fetal liver or cord blood derived HLA-DQ8+CD34+ cells as described generally in Example 7.

For HLA typing, DNA is isolated from CD34 negative fetal liver or cord blood cells using the DNeasy Blood & Tissue Kit (Qiagen) following isolation of CD34+ cells from these tissues. Sanger allele-level HLA typing is performed to determine the HLA type of the tissues. While the tissues are being typed, human fetal and cord blood CD34+ cells is frozen.

14-16 weeks post-transplantation, when HIS mice are fully reconstituted by human cells, they are euthanized for analysis. The percentages and absolute numbers of Clone 5+ thymocytes among double negative (CD1a+), including the CD7+ early thymocytes, CD69+ and CD69− double positive, CD4 single positive and CD8 single positive subsets are determined along with markers of negative selection (PD1,CCR7). Markers of Tregs (CD25 and CD127) are also included in the analysis in order to detect Treg lineage differentiation of thymocytes with this TCR in HLA-DQ8+ thymi. The detailed panel is shown in Table 5 below. Analysis is performed with Aurora Spectral flow cytometry.

Since the insulin peptide recognized by this TCR is expected to be produced by medullary TECs (mTECs), both positive selection of this TCR depends on the expression of HLA-DQ8 by the thymic epithelium. Therefore, the failure of positive selection of the HLA class II-restricted TCR Clone 5 in fetal pig thymus is observed.

However, in some cases there is a cross-reactive determinant produced in the SLAhh pig thymus that will be capable of positively selecting this TCR. In this case, it is determined whether or not negative selection of thymocytes with this TCR occurs in the pig thymus reconstituted with HLA-DQ8+CD34+ cells.

Preliminary data in HLA-DQ8+ human thymi suggest that HLA-DQ8 is required on CD34 cell derived APCs in order to negatively select this TCR (see FIG. 8). This may still occur in a pig thymus containing human HLA-DQ8+ APCs, since the insulin B(9-23) peptide is identical in the pig and human insulin molecules and may be picked up and presented by human APCs in the porcine thymus graft. Fluorochrome-labelled Clone 5 Vβ-specific mAb (Vβ21.3) is used to identify transgenic T cells and GFP will serve as a marker of origin from a transduced HSPC. GFP+ and GFP− thymocytes at each stage of thymic development provide internally-controlled comparisons of the level of selection of Tg and non-Tg T cells in each individual mouse. These studies, conducted as the transgenic pigs are being produced (Examples 3 and 4), provide a baseline against which to determine the effect of HLA-DQ8 transgenes in fetal pig thymus on selection of HLA DQ8-restricted human T cells in a pig thymus.

TABLE 4
HIS mice made with fetal human and fetal non-human (porcine) thymus
tissues

| Group | Thymic tissue | Clone 5 TCR-transduced CD34+ cells |
|---|---|---|
| 1 | Fetal human thymus | HLA-DQ8+ Fetal liver derived |
| | | (autologous) |
| 2 | Fetal pig thymus (SLAhh) | HLA-DQ8+ Fetal liver derived |
| 3 | Fetal pig thymus (SLAhh) | HLA-DQ8+ Cord blood derived |

TABLE 5
Panel to study selection of Clone 5+ T cells in thymus

| | |
|---|---|
| GFP | GFP |
| Vbeta 21.3 | APC |
| Mouse CD45 | V450 |
| Human CD45 | QDot800 |
| CD3 | BV786 |
| CD4 | V500 |
| CD8 | BV480 |
| CD69 | BV650 |
| CD1a | PerCP-efluor710 |
| CD5 | BV711 |
| PD1 | PE-Dazzle 594 |
| CD34 | BV785 |
| CD38 | PE-Cy7 |
| CD7 | PE-Cy5 |
| CD31 | BV605 |
| CCR7 | BV421 |
| CD45RA | APC-H7 |
| CD25 | AF700 |
| CD127 | BV570 |
| Viability | Zombie NIR Dye |

### Example 11—Comparison of Rejection of Allogeneic Human Skin Grafts of HIS Mice

To investigate the function of the human immune system in HIS mice generated with different thymi and CD34+ cells, their ability to reject allogeneic skin grafts is compared. To this end, HIS mice are generated by implanting fetal pig or human thymi and CB or fetal liver-derived CD34+ cells (Table 6) as described generally in Example 7.

14-16 weeks post-transplantation, split-thickness (2.3 mm) skin sample from allogeneic human donor is grafted on the lateral thoracic wall. Skin grafts are evaluated daily from day 7 onward to 4 weeks followed by at least one inspection every third day thereafter. Grafts are defined as rejected when less than 10% of the graft remains viable. HIS mice constructed with both types of thymus and CD34+ cells are able to reject allogeneic skin grafts.

TABLE 6
HIS mice made with fetal human and fetal non-human (porcine) thymus
tissues to determine their ability to reject allogeneic human skin grafts

| Group | Thymic tissue | CD34+ cells |
|---|---|---|
| 1 | Fetal human thymus | Fetal liver derived (autologous) |
| 2 | Fetal pig thymus (SLAhh) | Fetal liver derived |
| 3 | Fetal pig thymus (SLAhh) | CB derived |

### Example 12—Comparison of Human Cell Reconstitution with Non-Transgenic Vs HLA-A2 Transgenic Pig Thymi

As shown in Example 7, HIS mice generated with fetal pig thymus and cord blood-derived CD34+ cells have minor functional defects in T cells compared to HIS mice generated with fetal thymus and autologous fetal liver derived CD34+ cells, such as reduced HLA restricted antigen responses and thymic selection of TCR-transduced T cells. The major reason is that swine leukocyte antigen (SLA), rather than HLA, molecules mediate thymocyte positive selection in the pig thymus and only a small subset of these selected T cells will be sufficiently cross-reactive with human HLA to recognize peptide antigens presented by HLA of the CD34 cell donor-derived DCs. This model is optimized by using transgenic (Tg) fetal pig thymus that expresses common HLA molecules, including HLA-A2 and HLA-DQ8.

Using the HLA-A2 transgenic fetal pig thymus of Example 3, immune reconstitution and immune function are compared in HIS mice generated with non-transgenic vs HLA-A2 transgenic fetal pig thymi.

Using thymectomized NSG mice, two types of HIS mice using transgenic and nontransgenic fetal pig thymus plus CB CD34+ cells as described in Table 7 and as described generally in Example 7 are generated.

Following generation of these HIS mice, the mice are monitored as follows.

Monitor and compare human immune cell reconstitution in the two types of HIS mice by determining the rate of repopulation and peripheral blood concentrations of T, B and myeloid cell populations, including CD4 and CD8 T cells, naïve and memory CD4 and CD8 T cells, regulatory T cells (Tregs) and T follicular helper (Tfh) cells; B cell subsets, monocytes and DCs, including classical DCs (cDC1s and cDC2s) and plasmacytoid DCs (pDCs). Every 4 weeks following transplantation, peripheral blood from HIS mice are obtained and red blood cells are lysed with ACK buffer. Flow cytometric analysis of peripheral blood is performed to determine percentages and absolute numbers of each population. Absolute numbers of each population is calculated using counting beads. The percentages of mice achieving reconstitution in each group of HIS mice is also be determined. The panels used to study the immune cell populations are shown in Table 1.

Monitor and compare plasma immunoglobulin levels (IgM and IgG) by ELISA every 4 weeks following transplantation in the three types of HIS mice.

14-16 weeks post-transplantation, when HIS mice are expected to fully be reconstituted by human cells, half of the animals in each group are euthanized and the size, structure, cellularity and cell populations within peripheral blood, lymph nodes, spleen and thymus of all groups are compared. Flow cytometry panels to study immune cell populations are the same as shown in Table 1. A small piece of each lymphoid tissue, including spleen, lymph node and thymus, is used for histological studies to compare the structures of these tissues. Serum immunoglobulin levels (IgM and IgG) are measured by ELISA in all HIS mice. In addition, the function of human T cells in the periphery of each group of mice is compared using in vitro assays of proliferation, cytokine production and cytotoxicity in response to pan-TCR stimulation (anti-CD3/CD28 beads), alloantigen stimulation, xenoantigen stimulation and tetanus toxoid neoantigen stimulation. Proliferation is determined by CFSE cellular dye dilution. Production of cytokines, including IL-2 and IFN-γ, is assayed by intracellular staining. For alloantigen and xenoantigen stimulation, allogeneic human PBMCs and 3rd party pig PBMCs is used as stimulators. Isolated splenic T cells from HIS mice are labeled with CFSE and cocultured with irradiated stimulators at the ratio of 1:1 for 6 days. CFSE dilution of human CD4 and CD8 T cells is determined by flow cytometry. For tetanus toxoid neoantigen stimulation, DCs are generated using the CB CD34+ cells that are used for generation of HIS mice. CD34+ cells are cultured with human cytokines, including stem cell factor, GM-CSF and IL-4 for 13 days for differentiation into dendritic cells. CD34-derived DCs are pulsed with tetanus toxoid neoantigen and then matured by TNF-α and PGE2 followed by coculture with CFSE-labeled isolated splenic T cells for 7 days. Proliferated T cells are determined by flow cytometry. Monocytes are stimulated with LPS and production of TNF-α, IL-6 and IL-10 in supernatant is determined by ELISA.

The remaining HIS mice are monitored up to 30 weeks to observe the persistence of reconstitution of each lineage and to observe for the emergence of graft-vs-host/autoimmune disease. Mice are bled every 4 weeks to determined human cell engraftment. Starting from 20 weeks post-transplantation, mice are scored for graft-vs-host disease twice per week until week 30 using the scoring system shown Example 6. All analyses performed at this time point are the same as those at week 14-16.

Similar myeloid reconstitution is found between the groups Immune reconstitution and function may be enhanced in the recipients of HLA transgenic pig thymus.

TABLE 7
HIS mice made with HLA-A2-transgenic and non-transgenic fetal pig
thymus tissues

| Group | Thymic tissue | CD34+ cells |
|---|---|---|
| 1 | HLA- A2-transgenic fetal pig thymus | HLA-A2+ CB derived |
| 2 | Non-transgenic fetal pig thymus | HLA-A2+ CB derived |
| | (SLAhh) | |

### Example 13—Compare Tolerance of Human T Cells Developing in HLA-A2-Transgenic Fetal Pig Thymus to HLA-A2 Molecule

One major characteristic of human T cells developing in HIS generated with HLA-A2-transgenic fetal pig thymus is expected to be tolerance to HLA-A2, as HLA-A2-reactive T cells will be purged through negative selection by thymic epithelial cells expressing HLA-A2 and/or suppressed by Tregs selected by TECs expressing HLA-A2. To this end, tolerance of T cells developing in HLA-A2-Tg vs non-Tg fetal pig thymus to the human Tg HLA molecule is compared. HIS mice are generated using HLA-A2-CB CD34+ cells to eliminate the negative selection of HLA-A2-reactive T cells by CD34+ cell-derived APCs. Groups of HIS mice generated are shown in Table 8. 14-16 weeks post-transplantation, splenic and mature thymic T cells are isolated and tested in vitro for tolerance to HLA-A2, which we expect to observe only in recipients of the HLA-A2-Tg fetal pig thymus, using DCs derived from the donor pigs. DCs are generated from fetal pig liver leukocytes, which will be harvested at the time of fetal thymus harvest and frozen until use. Fetal liver leukocytes are cultured in porcine stem cell factor, GM-CSF and IL-4 for 13 days to differentiate them into DCs. These studies include Treg depletion to determine the impact of transgenic expression of HLA-A2 on Treg suppression of responses to HLA-A2

TABLE 8
HIS mice made with HLA-A2-Tg and non-Tg fetal pig thymus tissues
for comparison of tolerance of human T cells to HLA-A2 molecule

| Group | Thymic tissue | CD34+ cells |
|---|---|---|
| 1 | HLA-A2-Tg fetal pig thymus | HLA-A2− CB derived |
| 2 | Non-Tg fetal pig thymus (SLAhh) | HLA-A2− CB derived |

### Example 14—Compare Selection of an HLA-A2-Restricted TCR in HIS Mice Generated with Control Vs HLA-A2-Tg Fetal Pig Thymus

Selection of an HLA-A2-restricted TCR, MART1 is compared in HIS mice generated with non-Tg control vs HLA-A2-Tg fetal pig thymus. Sublethally irradiated thymectomized NSG mice are be injected with MART-1-transduced HLA-A2+CB CD34+ cells followed by implantation of non-Tg control or HLA-A2-Tg fetal pig thymus (Table 9).

TABLE 9
HIS mice made with non-Tg control or HLA-A2-Tg fetal pig thymus
tissues for study of thymic selection of MART-1 TCR positive T cells

| Group | | MART-1 TCR-transduced |
|---|---|---|
| CD34+ | Thymic tissue | cells |
| 1 | HLA-A2-Tg fetal pig thymus | HLA-A2+ CB derived |
| 2 | Non-Tg fetal pig thymus | HLA-A2+ CB derived |
| | (SLAhh) | |

14-16 weeks post-transplantation, when HIS mice are fully reconstituted by human cells, they are euthanized for analysis. The percentages and absolute numbers of MART-1+ thymocytes among double negative (CD1a+), including CD7+ early thymocytes, double positive, CD4 single positive and CD8 single positive subsets are determined along with other markers of negative selection (CD69, PD1,CCR7). It is expected to see enhanced positive selection of the HLA class I restricted TCR MART1 in HLA-A2+Tg fetal pig thymus. Fluorochrome-labelled MART1 tetramer is used to identify Tg T cells and GFP serves as a marker of origin from a transduced HSPC. GFP+ and GFP− thymocytes at each stage of thymic development provides internally controlled comparisons of the level of selection of Tg and non-Tg T cells in each individual mouse. The detailed panel is shown in Table 4 above. Analysis will be performed with the Aurora Spectral flow cytometer

MART1+ and negative CD8+ T cells in the periphery of each mouse (blood, spleen lymph nodes) are enumerated, hypothesizing that HLA-A2 resulting in increased positive selection in the pig thymus will result in export of greater numbers of MART1+ T cells to the periphery. The function of peripheral MART1+ cells is examined by labeling them with cell proliferation dye eFluor 450, incubating them with autologous DCs and added graded amounts of MART1 peptide, measuring proliferation and other markers of activation of GFP+ T cells.

### Example 15—Comparison of Rejection of Allogeneic Skin Grafts by HIS Mice Generated with HLA-A2-Tg Fetal Pig Thymus

To investigate the function of immune systems in HIS mice generated with HLA-A2-Tg thymi and CD34+ cells, the ability of HIS mice to reject allogeneic skin grafts is compared. To this end, HIS mice are be generated by implanting HLA-A2-Tg or non-Tg control fetal pig thymi and CB CD34+ cells to sublethally irradiated thymectomized NSG mice (Table 10). 14-16 weeks post-transplantation split-thickness (2.3 mm) skin samples from allogeneic human donors are grafted on the thoracic wall. Skin grafts are evaluated daily from day 7 onward to 4 weeks followed by at least one inspection every third day thereafter. Grafts are defined as rejected when less than 10% of the graft remains viable. Peripheral T cell function is more robust when the thymus and peripheral human APCs share an HLA molecule, resulting in more rapid graft rejection in the recipients of HLA-A2-Tg than control porcine thymic grafts.

TABLE 10
HIS mice made with HLA-A2-Tg and non-Tg fetal pig thymus tissues
to determine their ability to reject allogeneic human skin grafts

| Group | Thymic tissue | CD34+ cells |
|---|---|---|
| 1 | HLA-A2-Tg fetal pig thymus | HLA-A2+ CB derived |
| 2 | Non-Tg fetal pig thymus (SLAhh) | HLA-A2+ CB derived |

### Example 16—Comparison of Human Cell Reconstitution with Non-Tg Vs HLA-A2/DQ8− Tg Pig Thymi

When the HLA-A2/DQ8-Tg fetal pig thymus is available, immune reconstitution and immune function in HIS mice generated with non-Tg vs HLA-A2/DQ8− Tg fetal pig thyme is compared. Using thymectomized NSG mice, two types of HIS mice are generated using HLA-A2-Tg and HLA-A2/DQ8-Tg fetal pig thymus plus HLA-DQ8+CB CD34+ cells as described in Table 11. HLA-DQ8+CB CD34+ cells are used to generate HIS mice, as the presence of HLA-DQ8+ APCs in the periphery is required for optimal homeostasis of human T cells selected by HLA-DQ8. HLA-A2+DQ8+CD34+ cells are used in order to optimize immune function by having both a class I and a class II HLA allele shared by the thymus and peripheral APCs.

TABLE 11
HIS mice made with HLA-A2/DQ8-Tg and non-Tg fetal pig
thymus tissues for comparison of human cell reconstitution

| Group | Thymic tissue | CD34+ cells |
|---|---|---|
| 1 | HLA-A2/DQ8-Tg fetal pig thymus | HLA-A2/DQ8+ CB |
| derived | | |
| 2 | HLA-A2-Tg fetal pig thymus | HLA-A2/DQ8+ CB |
| derived | | |

Following generation of these HIS mice, the mice are monitored as follows:

Monitor and compare human immune cell reconstitution in the two types of HIS mice by determining the rate of repopulation and peripheral blood concentrations of T, B and myeloid cell populations, including CD4 and CD8 T cells, naïve and memory CD4 and CD8 T cells, regulatory T cells (Tregs) and T follicular helper (Tfh) cells; B cell subsets, monocytes and DCs, including classical DCs (cDC1s and cDC2s) and plasmacytoid DCs (pDCs). Every 4 cells are lysed with ACK buffer. Flow cytometric analysis of peripheral blood is performed to determine percentages and absolute numbers of each population. Absolute numbers of each population is calculated using counting beads. The percentages of mice achieving reconstitution in each group of HIS mice will also be determined. The panels used to study the immune cell populations are shown in Table 2.

Monitor and compare plasma immunoglobulin levels (IgM and IgG) by ELISA every 4 weeks following transplantation in the three types of HIS mice.

14-16 weeks post-transplantation, when HIS mice are expected to fully be reconstituted by human cells, half of the animals in each group are euthanized and the size, structure, cellularity and cell populations within peripheral blood, lymph nodes, spleen and thymus of all groups is compared. Flow cytometry panels to study immune cell populations are the same as shown in Table 2. A small piece of each lymphoid tissue, including spleen, lymph node and thymus, is used for histological studies to compare the structures of these tissues. Serum immunoglobulin levels (IgM and IgG) is measured by ELISA in all HIS mice. In addition, the function of human T cells in the periphery of each group of mice is compared using in vitro assays of proliferation, cytokine production and cytotoxicity in response to pan-TCR stimulation (anti-CD3/CD28 beads), alloantigen stimulation, xenoantigen stimulation and tetanus toxoid neoantigen stimulation. Proliferation is determined by CFSE cellular dye dilution. Production of cytokines, including IL-2 and IFN-γ, is assayed by intracellular staining. For alloantigen and xenoantigen stimulation, allogeneic human PBMCs and 3rd party pig PBMCs is used as stimulators. Isolated splenic T cells from HIS mice is labeled with CFSE and co-cultured with irradiated stimulators at the ratio of 1:1 for 6 days. CFSE dilution of human CD4 and CD8 T cells are determined by flow cytometry. For tetanus toxoid neoantigen stimulation, DCs are generated using the CB CD34+ cells that are used for generation of HIS mice. CD34+ cells are cultured with human cytokines, including stem cell factor, GM-CSF and IL-4 for 13 days for differentiation into dendritic cells. CD34-derived DCs are pulsed with tetanus toxoid neoantigen and then matured by TNF-α and PGE2 followed by co-culture with CFSE-labeled isolated splenic T cells for 7 days. Proliferated T cells will be determined by flow cytometry. Monocytes are stimulated with LPS and production of TNF-α, IL-6 and IL-10 in supernatant is determined by ELISA.

The remaining HIS mice are monitored up to 30 weeks to observe the persistence of reconstitution of each lineage and to observe for the emergence of graft-vs-host/autoimmune disease. Mice are bled every 4 weeks to determined human cell engraftment. Starting from 20 weeks post-transplantation, mice are scored for graft-vs-host disease twice per week until week 30 using the scoring system shown Example 8. All analyses performed at this time point will be the same as those at week 14-16.

### Example 17—Comparison of Tolerance to HLA-DQ8 of Human T Cells Developing in HLA-A2/DQ8-Tg Vs HLA-A2-Tg Fetal Pig Thymus

The tolerance of T cells developing in HLA-A2/DQ8-Tg vs non-Tg fetal pig thymus to the human Tg HLA-DQ8 molecule is compared. HIS mice are generated using HLA-DQ8−CB CD34+ cells to eliminate the negative selection of HLA-DQ8-reactive T cells by CD34+ cell derived APCs. Groups of HIS mice generated for this task are shown in Table 12. 14-16 weeks posttransplantation, splenic and mature thymic T cells are isolated and tested in vitro for tolerance to HLA-DQ8, which it is expected to observe only in recipients of the HLA-A2/DQ8-Tg fetal pig thymus, using DCs derived from the donor pigs. DCs are generated from fetal pig liver leukocytes, which will be harvested at the time of fetal thymus harvest and frozen until use. Fetal liver leukocytes are cultured in porcine stem cell factor, GM-CSF and IL-4 for 13 days to differentiate them into DCs. These studies include Treg depletion, as the presence of HLADQ8 on TECs may permit the positive selection of Tregs with these specificities.

TABLE 12
HIS mice made with HLA-A2/DQ8-Tg and HLA-A2-Tg fetal pig
thymus tissues for comparison of tolerance of human T cells to
HLA-DQ8 molecule

| Group | Thymic tissue | CD34+ cells |
|---|---|---|
| 1 | HLA-A2/DQ8-Tg fetal pig thymus | HLA-A2+DQ8− CB |
| derived | | |
| 2 | HLA-A2-Tg fetal pig thymus | HLA-A2+DQ8− CB |
| derived | (SLAhh) | |

### Example 18—Compare Selection of an HLA-DQ8-Restricted TCR in HIS Mice Generated with Control Vs HLA-A2/DQ8-Tg Fetal Pig Thymus

Selection of an HLA-DQ8-restricted TCR (Clone 5) in HIS mice generated with non-Tg control vs HLA-A2/DQ8-Tg fetal pig thymus is compared. Sublethally irradiated thymectomized NSG mice are be injected with Clone 5-transduced CB CD34+ cells following by implantation of non-Tg control or HLA-A2/DQ8-Tg fetal pig thymus (Table 13).

TABLE 13
HIS mice made with non-Tg control or HLA-A2/DQ8-Tg fetal pig thymus
tissues for study of thymic selection of MART-1 TCR positive T cells

| | | Clone 5 TCR-transduced |
|---|---|---|
| Group | Thymic tissue | CD34+ cells |
| 1 | HLA-A2/DQ8-Tg fetal pig thymus | HLA-DQ8+ CB derived |
| 2 | Non-Tg fetal pig thymus (SLAhh) | HLA-DQ8+ CB derived |

14-16 weeks post-transplantation, when HIS mice are fully reconstituted by human cells, HIS mice are euthanized for analysis. The percentages and absolute numbers of Clone 5+ thymocytes among double negative (CD1a+), including the CD7+ early thymocytes, CD69+ and CD69− double positive, CD4 single positive and CD8 single positive subsets are determined along with markers of negative selection (PD1,CCR7). Markers of Tregs (CD25 and CD127) are also evaluated in order to detect Treg lineage differentiation of thymocytes with this TCR in HLA-DQ8+ thymi. The detailed panel is shown in Table 5 above. Analysis will be performed with Aurora Spectral flow cytometry. Since the insulin peptide recognized by this TCR is expected to be produced by medullary TECs (mTECs), negative selection of this TCR is expected to depend on the expression of HLA-DQ8 by the thymic epithelium. It is expected to see enhanced positive selection of the HLA class II-restricted TCR Clone 5 in HLA-A2/DQ8-Tg fetal pig thymus compared to non-Tg pig thymi. Preliminary data in HLA-DQ8+ human thymi suggest that, in addition to expression on TEC, HLA-DQ8 is required on CD34 cell-derived APCs in order to negatively select this TCR (see FIG. 9). Thus, the use of HLA-DQ8+CB CD34+ cells to generate HIS mice will also allow the study of negative selection of Clone5+ T cells. Fluorochrome-labelled Clone 5 Vβ-specific mAb (V1321.3) are used to identify Tg T cells and GFP serves as a marker of origin from a transduced HSPC. GFP+ and GFP− thymocytes at each stage of thymic development provide internally-controlled comparisons of the level of selection of Tg and non-Tg T cells in each individual mouse.

### Example 19—Compare Rejection of Allogeneic Skin Grafts by HIS Mice Generated with HLA-A2/DQ8 Tg Fetal Pig Thymus

To investigate the function of immune systems in HIS mice generated with HLA-A2/DQ8-Tg thyme and CD34+ cells, are compared for their ability to reject allogeneic skin grafts. To this end, HIS mice are generated by implanting HLA-A2/DQ8-Tg or non-Tg control fetal pig thymi and CB CD34+ cells (Table 10). 14-16 weeks post-transplantation, split-thickness (2.3 mm) skin samples from allogeneic human donors are grafted on the lateral thoracic wall. Skin grafts are evaluated daily from day 7 onward to 4 weeks followed by at least one inspection every third day thereafter. Grafts are defined as rejected when less than 10% of the graft remained viable.

TABLE 14
HIS mice made with HLA-A2/DQ8-Tg and non-Tg fetal pig thymus
tissues to determine their ability to reject allogeneic human skin grafts

| Group | Thymic tissue | CD34+ cells |
|---|---|---|
| 1 | HLA-A2/DQ8-Tg fetal pig thymus | HLA-DQ8+ CB derived |
| 2 | Non-Tg fetal pig thymus (SLAhh) | HLA-DQ8+ CB derived |

### REFERENCES

Cheng, et al. Expression and function of HLA-DQ8 (DQA1*0301/DQB1*0302) genes in transgenic mice. Eur J Immunogenet. 1996; 23(1):15-20.
Hongo, et al. Porcine thymic grafts protect human thymocytes from HIV-1-induced destruction. J Infect Dis. 2007; 196(6):900-910.
Kalscheuer, et al. A model for personalized in vivo analysis of human immune responsiveness. Science Translational Medicine. 2012; 4(125):125-130.
Kalscheuer, et al. Xenograft tolerance and immune function of human T cell developing in pig thymus xenografts. Journal of Immunology. 2014; 192(7):3442-3450.
Khosravi Maharlooei, et al. Rapid thymectomy of NSG mice to analyze the role of native and grafted thymi in humanized mice European J. of Immunology 2019; 50(1): 138-141.
Kotsiou, et al. Properties and applications of single-chain major histocompatibility complex class I molecules. Antioxid Redox Signal. 2011; 15(3):645-655.
Lan, et al. Induction of human T cell tolerance to porcine xenoantigens through mixed hematopoietic chimerism. Blood. 2004; 103:3964-3969.
Lan, et al. Reconstitution of a functional human immune system in immunodeficient mice through combined human fetal thymus/liver and CD34+ cell transplantation. Blood. 2006; 108(2):487-492.
Melkus, et al. Humanized mice mount specific adaptive and innate immune responses to EBV and TSST-1. Nat Med. 2006; 12(11):1316-1322.
Nikolic, et al. Normal development in porcine thymus grafts and specific tolerance of human T cells to porcine donor MHC. J. Immunol. 1999; 162:3402-3407.
Pascolo, et al. HLA-A2.1-restricted education and cytolytic activity of CD8(+) T lymphocytes from beta2 microglobulin (beta2m) HLAA2.1 monochain transgenic H-2db beta2m double knockout mice. J Exp Med. 1997; 185(12):2043-2051.
Shimizu, et al. Comparison of human T cell repertoire generated in xenogeneic porcine and human thymus grafts. Transplantation. 2008; 86(4):601-610.
Zhao, et al. Positive and negative selection of functional mouse CD4 cells by porcine MHC in pig thymus grafts. J. Immunol. 1997; 159:2100-2107.
Zhao, et al. Pig MHC mediates positive selection of mouse CD4+ T cells with a mouse MHC-restricted TCR in pig thymus grafts. J. Immunol. 1998; 161:1320-1326.

1. A transgenic swine, comprising one or more nucleotide sequences encoding one or more HLA I polypeptides and/or one or more HLA II polypeptides inserted into one or more native SLA loci of the pig genome.

2. The transgenic swine of claim 1, wherein the one or more nucleotide sequences encode HLA I polypeptides inserted into a native SLA I locus.

3. The transgenic swine of claim 2, wherein the SLA I locus is selected from the group consisting of SLA-1 and SLA-2.

4. The transgenic swine of claim 2, wherein the HLA I polypeptides comprise HLA-A2 fused to human beta-2 microglobulin (B2M).

5. The transgenic swine of claim 2-4, wherein the one or more nucleotide sequences are inserted behind a native SLA I promoter.

6. The transgenic swine of claim 2-4, wherein the one or more nucleotide sequences are inserted at the intron 1/exon 2 junction of the SLA I locus.

7. The transgenic swine of claims 2-6, wherein the one or more nucleotide sequences further encode HLA II polypeptides inserted into the native SLA-DQα locus.

8. The transgenic swine of claim 1, wherein the one or more nucleotide sequences encode HLA II polypeptides inserted into the native SLA-DQα locus.

9. The transgenic swine of claims 7-8, wherein the HLA II polypeptides comprise the HLA-DQ8 polypeptides.

10. The transgenic pig of claim 10, wherein the HLA-DQ8 polypeptides are targeted to the native SLA-DQα locus through a bicistronic vector encoding HLA-DQ8 (HLA-DQA1:03:01:01 and HLA-DQB1:03:02:01).

11. The transgenic swine of claim 10, wherein the bicistronic vector further comprises a high-efficiency IRES element.

12. The transgenic swine of claims 7-11, wherein the one or more nucleotide sequences encoding the HLA II polypeptides are inserted behind the native SLA DQa promoter.

13. The transgenic swine of claims 7-11, wherein the one or more nucleotide sequences encoding the HLA II polypeptides are inserted at the intron 1/exon 2 junction of the SLA DQa locus.

14. The transgenic swine of claim 1, wherein the HLA I polypeptides are selected from the group consisting of HLA-A, HLA-A2, HLA-B, HLA-C, HLA-E, HLA-F and HLA-G, and wherein the HLA II polypeptides are selected from the group consisting of HLA-DP, HLA-DM, HLA-DO, HLA-DQ, and HLA-DR.

15. A method of xenotransplantation of thymic tissue into a subject in need thereof, comprising the introduction of thymic tissue from the transgenic swine according to any of claims 1-14 into the subject.

16. A method of recovering or restoring impairment of the function of the thymus in a subject in need thereof, comprising the introduction of thymic tissue from the transgenic swine according to any of claims 1-14 into the subject.

17. A method of reconstituting T cells in a subject in need thereof, comprising the introduction of thymic tissue from the transgenic swine according to any of claims 1-13 into the subject.

18. The methods of claims 15-17, wherein the subject is a human.

19. The method of claims 15-18, wherein the transgenic swine comprises HLA polypeptides derived from the subject.

20. A method of producing a transgenic swine of any of claims 1-14, comprising administering at least one targeting vector and at least one CRISPR-Cas9 plasmid into a swine cell, wherein the targeting vector comprises one or more nucleotide sequences encoding one or more HLA I polypeptides and/or one or more HLA II polypeptides.

21. The method of claim 20, wherein the one or more nucleotide sequences encoding one or more HLA I polypeptides and/or one or more HLA II polypeptides derive from a specific individual subject.

22. A method of generating a human immune system (HIS) mouse, comprising thymectomizing the mouse and introducing swine fetal thymic tissue and human CD34+ cells into the mouse.

23. The method of claim 22, wherein the human CD34+ cells are fetal or adult.

24. The method of claim 22, wherein the human CD34+ cells are derived from cord blood.

25. A method of generating a human immune system (HIS) mouse, comprising thymectomizing the mouse and introducing swine fetal thymic tissue, wherein the fetal thymic tissue is derived from the transgenic swine of claims 1-14.
